(19) United States
(12) Patent Application Publication
(10) Pub. No.: US 2017/0154163 A1
(43) Pub. Date: Jun. 1, 2017

(54) CLINICALLY RELEVANT SYNTHETIC LETHALITY BASED METHOD AND SYSTEM FOR CANCER PROGNOSIS AND THERAPY

(71) Applicant: Ramot at Tel-Aviv University Ltd., Tel Aviv (IL)

(72) Inventors: Livnat JERBY ARNON, Tel Aviv (IL); Eytan RUPPIN, North Bethesda, MD (US)

(21) Appl. No.: 15/366,434

(22) Filed: Dec. 1, 2016

Related U.S. Application Data
(60) Provisional application No. 62/261,325, filed on Dec. 1, 2015.

(51) Int. Cl. G06F 19/00; G06F 17/18; A61K 31/502; A61K 31/7048; G06F 19/24; G06F 19/18

(52) U.S. Cl. CPC G06F 19/3437; G06F 19/24; G06F 19/18; A61K 31/502; A61K 31/7048; G06F 17/18

(57) ABSTRACT

Systems and methods for identifying clinically relevant Synthetic Lethal interactions SLi by analyzing large and diverse cohorts of clinically relevant cancer data, utilizing a data driven approach, termed SLICK, are provided. Further provided are system and methods of utilizing SLICK to uncover therapeutic possibilities in cancer.

### FIELD OF THE INVENTION

The invention is in the field of bioinformatics, cancer research, personalized medicine, cancer therapy and cancer drug development. The invention provides systems and methods for identifying and utilizing clinically relevant synthetic lethal interaction (SLi) networks for predicting drug responses and selection of candidate drugs and drug combinations for cancer therapy.

### BACKGROUND OF THE INVENTION

The rapidly accumulating data obtained from cancer clinical samples has revolutionized cancer research. One of the key objectives is to systematically map between the genomic and molecular characteristics of tumors and their responses to various drugs. One way by which to tackle this and realize the potential of cancer pharmacogenomics is based on the concept of Synthetic lethal interactions (SLi). SLi describe the relationship between two genes whereby an individual inactivation of either gene results in a viable phenotype, while their combined inactivation is lethal^{1,2}. SLi have been considered as a potential basis for developing selective anticancer drugs³⁻⁵. Such drugs are aimed at inhibiting the Synthetic Lethal (SL) partner of a gene that is inactive in the cancer cells. Indeed, as 90% or more of cancer predisposing mutations result in a loss of protein function, by identifying SLi these genomic alterations can be exploited for developing and improving cancer treatments.

SLi are conventionally referred to as symmetric. However, considering the sequential nature of genomic alterations in cancer, the alteration of one gene induces cellular changes that can affect the response to subsequent alterations. The inactivation of gene A may lead to the induced essentiality³ of gene B, while the inactivation of B may not render A essential. A particularly interesting class of such asymmetric SLi—(termed regulatory SLi)—captures tumorigenic events where following the inactivation of a tumor suppressor cancer cells become, over time, dependent on specific oncogenic transformations⁸. Additionally, some SLi are cumulative, such that the more (possibly partially) inactive SL-partners a gene has, the more lethal its inhibition is. Cumulative SLi may arise due to cumulative haploinsufficiency and tumor heterogeneity⁹.

Considerable work has been devoted to identifying SLi in cancer—both experimentally^{2,3,10-17} and computationally¹⁸⁻²⁷. Various machine learning methodologies have been applied to predict SLi in yeast, worm, and fly based on known interactions, using gene expression, phenotype data, protein-protein interactions, and functional annotations of genes^{18,21-23,28,29}. Cancer SLi have been inferred by utilizing yeast SLi²⁴, metabolic models, and evolutionary characteristics^{23,25,26}. The DAta-mining SYnthetic-lethality identification pipeline (DAISY) for identifying cancer SLi was recently published^{30,31}. DAISY is based on the observation that cells with a joint inactivation of two SL genes are selected against. Following this rationale, DAISY identifies gene pairs as candidate SL if their inactivation patterns are mutually exclusive across thousands of cancer genomic profiles. Additional studies have harnessed similar principles and mined cancer data to identify SLi³²⁻³⁴ and oncogenic pathway modules^{35,36}.

US patent application no. 2015/0331992 of the inventors of the present invention, discloses a method, termed DAISY for cancer prognosis and treatment based on synthetic lethality obtained predominantly from in-vitro data. Jerby-Arnon et al. discloses “Harnessing Synthetic Lethality to Predict Clinical Outcomes of Cancer Treatment” (A seminar presented at the Rappaport Faculty of Medicine, Technion—Israel Institute of Technology on Oct. 22, 2015, 64 pages).

There is still an unmet need to provide optimized and clinically-relevant methods for precise and personalized cancer prognosis and treatment.

### SUMMARY OF THE INVENTION

The present invention provides, in embodiments thereof, systems and methods for identification and creation of Synthetic Lethal interactions (SLi) and networks and uses thereof to uncover therapeutic possibilities and drug response, for example, in cancer conditions.

According to some embodiments, the systems and methods disclosed herein utilize a data-driven computational approach, termed herein SLICK, that identifies and exploits Synthetic Lethal interactions (SLi) to uncover therapeutic possibilities in cancer. SLi occur when the inactivation of two non-essential genes is lethal, and can be utilized to selectively target cancer cells. SLICK identifies clinically relevant SLi by analyzing large and diverse cohorts of clinically relevant cancer data based on the observation that cancer cells are shaped by clonal selection, which is modulated by cancer therapy.

The SLICK approach disclosed in the present invention was used to generate the first clinically-derived pan-cancer SLi-network, comprising both protein-coding and micro-RNA genes. The network successfully identified known gene essentiality and drug responses. Furthermore, advantageously, the, SLi network identified utilizing the systems and methods disclosed herein, was used to identify novel drug repurposing options. In exemplary embodiments, the methods and systems disclosed herein identifies an effective treatment against BRCA-deficient tumors, and discovers drugs synergistic with inhibition of the enzyme poly ADP ribose polymerase (PARP).

In some embodiments, the clinical SLi-network of the present invention is the first SLi-network that successfully predicts the response of cancer patients to chemotherapies and targeted therapies, outperforming commonly used drug response sequence-based biomarkers and gene expression signatures.

According to one aspect, there is provided a system for identifying clinically relevant synthetic lethal interactions (SLi) of pairs of genes from cancer patients, the system comprising:
a non-transitory computer readable memory having stored thereon datasets comprising data related to multiple genes in said cancer patients, and a processing circuitry configured to recursively:
   i. assign SLi-p values to each ordered gene pair obtained from a cancer patient data (dataset x-gene x);
   ii. omit unlikely SLi to obtain significant SLi-p values;
   iii. perform simulated annealing (SA) to optimize the network ability to predict a clinical drug response;
   iv. repeat step (iii) N times; and
   v. merge the solutions obtained in (iv) to construct a final SL network according to all N solutions.

In some embodiments, the SLi-p-value denote the likelihood of a gene pair of being synthetic Lethal (SL). In some embodiments, the SLi-p-value is determined utilizing data-driven inference procedures selected from: genomic Survival of the Fittest (gSoF); Clinical survival analyses, and/or Correlated expression.

In some embodiments, step (iii) comprises eliminating false positive predictions emerged in steps (i.) or (ii.).

In some embodiments, N is at least 200. In some embodiments, N is 500-2000. In some embodiments, N is at least 1000. According to yet other embodiments, N is 1000-5000.

In some embodiments, weight or strength of a given SLi in the final SL network is the fraction of solutions in which it appeared.

In some embodiments, the cancer patient data is selected from activity profile of the genes, essentiality profile of the genes, expression profile of the genes, treatment, response to treatment, prognosis, survival, or combinations thereof. In some embodiments, the activity profile of the genes comprises Somatic Copy Number of Alterations (SCNA), germline Copy-Number Variations (CNV), DNA methylation, histone methylation, somatic mutations, germline mutations or combinations thereof, obtained from at least one cancer patient.

In some embodiments, the processing circuitry is further configured to determine an occurrence selected from the group consisting of:
a. response of cancer cells to the inhibition of a gene product;
b. survival of a subject having cancer;
c. response of cancer cells to a specific drug; and
d. ranking of cancer treatments for a specific subject having cancer;
wherein the determination comprising applying the identified SL-network on a genomic profile of cells, wherein the genomic profile of cells is obtained from at least one cancer patient.

In some embodiments, the system may further allow predicting one or more of: clinical response of a cancer patient to a drug; drug repurposing, drug combinations, or combinations thereof.

According to another aspect, there is provided a system for predicting clinical anti-cancer drug response utilizing a clinically relevant synthetic lethal interactions (SLi) network of pairs of genes from cancer patients, the system comprising:
a non-transitory computer readable memory having stored thereon datasets comprising data related to multiple genes in said cancer patients, and a processing circuitry configured to recursively:
i. assign SLi-p values to each ordered gene pair obtained from a cancer patient data (dataset x-gene x);
ii. omit unlikely SLi to obtain significant SLi-p values;
iii. perform simulated annealing (SA) to optimize the network ability to predict a clinical drug response;
iv. repeat step (iii) N times;
v. merge the solutions obtained in (iv) to construct a clinically relevant synthetic lethal interactions (SLi) network according to all N solutions;
vi. integrating a SLi network of step (v.) 1 with a gene expression profile of at least one subject's tumor;
vii. predicting the response of said at least one subject tumor to a specific drug as proportional to the number of underexpressed SL-partners the specific drug target(s) has in the subject's tumor;
viii. classifying the subjects, based on step (vii) as responders or non-responders to the specific treatment said subjects received, and providing a computed logrank p-value to examine whether the responders outlived the non-responders;
ix. computing a control logrank p-value using randomly shuffled drug-patient mappings; and
x. calculating the ratio between the p-values of (viii) and (ix), wherein said ratio denotes the ability of the SL-network to specifically predict drug response while controlling for drug-independent patient survival rates.

In some embodiments, the SLi-p-value denote the likelihood of a gene pair of being synthetic Lethal (SL). In some embodiments, the SLi-p-value is determined utilizing data-driven inference procedures selected from: genomic Survival of the Fittest (gSoF); Clinical survival analyses, and/or Correlated expression.

In some embodiments, step (iii) comprises eliminating false positive predictions emerged in steps (i.) or (ii.).

In some embodiments, N is at least 200. In some embodiments, N is 500-2000. In some embodiments, N is at least 1000. According to yet other embodiments, N is 1000-5000.

In some embodiments, analyzing includes analyzing at least one data type selected from the group consisting of: Somatic Copy Number Alterations (SCNA), gene expression, somatic mutation profiles, treatment information, and survival data collected from clinical samples of subjects having cancer.

In some embodiments, analyzing includes analyzing at least one additional data type. In some embodiments, the at least one additional data type is selected from the group consisting of: single-cell gene expression data, proteomics, protein modifications, and epigenetic alterations.

In some embodiments the system further include predicting drug repurposing and/or drug combinations useful in treating a subject's cancer condition.

The present invention provides, according to another aspect, a method of generating a network of clinically relevant synthetic lethal interactions (SLi) of pair of genes, the method comprising:
i. a data-mining step—integrative genome-wide analyses of molecular omics data and patient survival data obtained from subjects having cancer, examining for each pair of genes whether it exhibits the molecular and clinical patterns characteristic of SL-partners, thereby generating an initial draft SL-network; and
ii. a network optimization step of eliminating false positive predictions that may have emerged in the first step by optimizing the network ability to predict clinical drug response.

Detailed description of the methodologies of performing the data-mining and network optimization steps is disclosed below.

According to this aspect, the data-mining step comprises computing for each pair of genes a set of SLi-p-values that denote its likelihood of being SL, by employing three types of data-driven inference procedures: i. genomic Survival of the Fittest (gSoF); ii. Clinical survival analyses; and iii. Correlated expression. Each interface procedure considers the data collected for a specific cancer type in a specific study, resulting in the assignment of a few dozen SLi-p-values to each ordered pair of genes, to provide an initial draft network comprising gene pairs with an array of overall statistically significant SLi-p-values.

According to this aspect, the optimization step prunes the initial draft network and improves its ability to predict clinical drug response by employing an optimization heuristic called Simulated Annealing (SA) to examine different ways by which the SLi-p-values can be combined to discriminate between true and false predictions of SLi, and selecting the setting that obtains the most predictive and thus clinically-relevant network according to the clinical drug response (training) data.

According to some embodiments, the optimizing process is performed at least about 1,000 times using different subsets of the training data, resulting in at least about 1,000 networks or solutions, and then aggregates the different solutions into a weighted network such that the weight or strength of a given SLi is the fraction of solutions in which it appeared.

According to some embodiments the SL-network generated is used to predict clinical response of a cancer patient to a drug.

According to some embodiments, the method of generating a network of clinically relevant synthetic lethal interactions (SLi) of pair of genes comprises one or more of the steps described in FIGS. 1A-1C:
i. assigning SLi-p values to each ordered gene pair obtained from a cancer patient data (dataset x-gene x);
ii. omitting unlikely SLi to obtain significant SLi-p values;
iii. performing simulated annealing to optimize the network ability to predict clinical drug response;
iv. repeating step (iii) N times; and
v. merging the solutions obtained in step iv thereby constructing a final SL network according to all N solutions.

According to some embodiments N is at least 200. According to some embodiments, N is 500-2000. According to yet other embodiments, N is at least 1000. According to yet other embodiments N is 1000-5000.

According to another aspect, the present invention provides a method of predicting clinical drug response, the method comprising the steps:
i. integrating the network (of clinically relevant synthetic lethal interactions (SLi) of pair of genes) with the gene expression profiles of the patients' tumors;
ii. predicting the response of a patient to a specific drug in an unsupervised, straight forward manner as proportional to the number of underexpressed SL-partners the specific drug target(s) has in the patient's tumor;
iii. classifying the patients, based (ii) as responders and non-responders to the specific treatment they received, and providing a computed logrank p-value to examine whether the responders outlived the non-responders;
iv. computing a control logrank p-value using randomly shuffled drug-patient mappings; and
v. calculating the ratio between the p-values of (iii) and (v), wherein the ratio denotes the ability of the SL-network to specifically predict drug response while controlling for drug-independent patient survival rates.

According to some embodiments, the method comprises analyzing at least one data type selected from the group consisting of: Somatic Copy Number Alterations (SCNA), gene expression, somatic mutation profiles, treatment information, and/or survival data collected from clinical samples of subjects having cancer. Each possibility is a separate embodiment.

According to some embodiments, the analysis comprises data of Somatic Copy Number Alterations (SCNA), gene expression, somatic mutation profiles, treatment information, and/or survival data, collected from clinical samples of subjects having cancer. Each possibility is a separate embodiment.

According to some embodiments, the method comprises analyzing at least one additional data type. According to some embodiments, the at least one data type is selected from the group consisting of: single-cell gene expression data, proteomics, protein modifications, and epigenetic alterations. Each possibility is a separate embodiment.

The present invention provides, according to another aspect, a SL-network of clinically relevant synthetic lethal interactions (SLi) of pair of genes obtained by the systems and methods described above.

According to some embodiments, the SL-networks generated according to the present invention are applied, using method and systems described herein, to identify candidates for drug repurposing and drug combinations

According to yet other embodiments, the SL-networks, methods and systems according to the present invention are used to detect a subpopulation of responsive patients.

According to other embodiments, the SL-networks, methods and systems of the present invention are used for patients with tumors that do not bear specific oncogenic mutations.

According to other embodiments, the SL-networks, methods and systems of the present invention are used for identifying drug candidates and/or drug combinations for treating heterogeneous tumors and avoiding the emergence of drug resistance.

According to some embodiments the SL-network may be used to provide a research platform to bridge the gap between experimental models and the clinic by providing for example gene candidates for in-vitro assays, transgenic and knockout animals and agents for use as controls in screening assays.

According to some embodiments, the SL-network may be used for identification of SLi involving long-non-coding RNA and other non-coding regulatory genomic elements as enhancers and silencers.

According to yet other embodiments, the SL-network may be used for identifying cancer type-specific SL-networks.

The present invention provides, according to another aspect, a system for identifying clinically relevant synthetic lethal interactions (SLi) of pair of genes from cancer patients, the system comprising:
a non-transitory computer readable memory having stored thereon datasets comprising data related to multiple genes in said cancer patients, and a processing circuitry configured to recursively:
   i. assign SLi-p values to each ordered gene pair obtained from a cancer patient data (dataset x-gene x);
   ii. omit unlikely SLi to obtain significant SLi-p values;
   iii. perform simulated annealing (SA) to optimize the network ability to predict clinical drug response;
   iv. repeat step (iii) N times; and
   v. merge the solutions obtained in step (iv) to construct a final SL network according to all N solutions.

According to some embodiments, the SLi-p value denote that likelihood of the gene pair of being synthetic Lethal (SL). According to some embodiments, the SLi-p-value is determined utilizing data-driven inference procedures selected from: genomic Survival of the Fittest (gSoF); Clinical survival analyses; and iii) Correlated expression. In some embodiments, each interface procedure considers the data collected for a specific cancer type in a specific study, resulting in the assignment of a several SLi-p-values to each ordered pair of genes, to provide an initial draft network comprising gene pairs with an array of overall statistically significant SLi-p-values.

According to some embodiments, step (iii) comprises eliminating false positive predictions that may have emerged in the analysis. According to some embodiments, the optimization step prunes the initial draft network and improves its ability to predict clinical drug response by employing the optimization heuristic, Simulated Annealing (SA), to examine different ways by which the SLi-p-values can be combined to discriminate between true and false predictions of SLi, and selecting the setting that obtains the most predictive and thus clinically-relevant network according to the clinical drug response (training) data

According to some embodiments, the cancer patient data is selected from activity profile of the genes, essentiality profile of the genes, expression profile of the genes, treatment, response to treatment, prognosis, survival or combinations thereof.

According to some embodiments, activity profile of the genes comprises Somatic Copy Number of Alterations (SCNA), germline Copy-Number Variations (CNV), DNA methylation, histone methylation, somatic mutations, germline mutations or combinations thereof, obtained from at least one cancer patient.

According to some embodiments, the processing circuitry is further configured to determine an occurrence selected from the group consisting of:
i. response of cancer cells to the inhibition of a gene product;
ii. survival of a subject having cancer;
iii. response of cancer cells to a specific drug; and
iv. ranking of cancer treatments for a specific subject having cancer;
comprising applying the identified SL-network on a genomic profile of cells, wherein the genomic profile of cells is obtained from at least one cancer patient.

According to some embodiments, there is provided a method of treating a disease or condition in a subject in need thereof, wherein the treatment regime (i.e., the type of drug, combination of drugs, dosage regime, administration mode, and the like) is determined according to the methods and systems disclosed herein.

In some embodiments, the method comprises generating a suitable SLi network and applying the generated network on a genomic profile of cells obtained from the subject.

According to yet another aspect, there is provided a method of treating a subject having a BRCA1/2-deficient cancer comprising administering to said subject a combination therapy comprising the PARP1/2/3 inhibitor olaparib and the topoisomerase II (TOP2A) inhibitor etoposide.

According to the some embodiments, such administration may be performed in a combined composition or in separate compositions administered together or at separate times.

According to some embodiments, the cancer is selected from the group consisting of: oral cancer, breast cancer and pancreatic cancer. According to particular embodiments, the cancer is a pancreatic cancer.

According to some embodiments, the method comprises administration of olaparib and etoposide in conjugation with surgery, radio- or chemotherapy.

According to some embodiments, the method resulted in increasing the progression free survival of a subject having cancer, increasing the duration of response of a subject having cancer, preventing or inhibiting development of metastasis in a patient having cancer, and/or preventing tumor recurrence.

In another embodiments, the present invention provides a pharmaceutical composition comprising olaparib and etoposide and at least one carrier, excipient, diluent or salt.

Further embodiments and the full scope of applicability of the present invention will become apparent from the detailed description given hereinafter. However, it should be understood that the detailed description and specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE FIGURES

FIGS. 1A-1C. Graphical demonstration of steps and patterns implemented by the SLICK method. FIG. 1A The gSoF-SLi-pattern: The activity level of gene B is significantly higher in A-deficient samples compared to samples in which A is active. FIG. 1B—The clinical-survival-SLi-pattern: the inactivity of gene B is a marker for patient survival specifically in the subpopulation of patients with A-deficient tumors. FIG. 1C—workflow low the landscape of the SLICK method.

FIGS. 2A-2B. The clinical SL-network predicts gene essentiality and drug response in cancer cell lines. FIG. 2A—Graphs showing the performances of the clinical SL-network compared to that of DAISY when applied to predict gene essentiality in 129 cancer cell lines, according to two large gene essentiality screens: Achilles³⁸ (left), and Marcotte (right)³⁷. The prediction accuracy is quantified according to the AUC measure). FIGS. 2B-2E—The predicted vs. experimental drug efficacies according to (from right to left): Cancer Genome Project (CGP)³⁹, Cancer Therapeutics Response Portal (CTRP)⁴⁰ and Cancer Cell Line Encyclopedia (CCLE)⁴¹ collections. Rs and Rp denote the Spearman and Pearson correlation coefficients, respectively. All correlations were highly significant (P<1e-30).

FIGS. 3A-3F. Experimental validation of the clinical-SL-network. FIG. 3A-3D)—In-vitro large-scale validation of the SL-network as a drug response and drug repurposing predictor. FIG. 3A—The Receiver Operating Characteristic (ROC)-curve obtained when applying the clinical SL-network to predict growth-arresting drugs under normoxia and hypoxia in oral cancer. FIG. 3B—The predicted efficacy of drugs in oral cancer shown for drug-condition pairs with high, moderate and low experimentally measured growth inhibition (GI %). FIG. 3C The ROC-curve obtained when applying the network to predict growth-arresting drugs in a panel of breast cancer cell lines. FIG. 3D The predicted efficacy of drugs in different breast cancer cell lines, shown for drug-cell line pairs with high and low experimentally measured GI %. FIGS. 3E-3F: Validating the SLi BRCA-TOP2A and PARP3-HDAC2. FIG. 3E—The GI % effect of etoposide in the BRCA-deficient Capan-1 cells and in the BRCA-wild type pancreatic cell lines (AsPC1 and BxPC3) and patient derived xenograft cultures (X50 and X57). FIG. 3F—The GI % observed in Capan-1 when administering olaparib and vorinostat separately or in combination. In FIGS. 3E-3F, the mean values are shown; error bars represent standard deviation.

FIGS. 4A-4E. The clinical-SL-network predicts treatment outcomes in cancer patients. FIG. 4A—The survival Kaplan Meier (KM)-plots of The Cancer Genome Atlas (TCGA) patients that were predicted as responders (upper line in all plots) and non-responders (lower line in all plots) to the treatment they received, shown when considering all drugs in the dataset. FIGS. 4B-4D—The DRSF rates of breast cancer patients that were predicted as responders (upper line in all plots) and non-responders (lower line in all plots) to the treatment they received: FIGS. 4B-4C—HER2-negative breast cancer patients treated with taxane-anthracycline chemotherapy; FIG. 4D—estrogen receptor positive breast cancer patients treated with tamoxifen54. In FIGS. 4A-4D, In parenthesis next to the name of each group are the number of patients and the number and fraction of deaths in that group. FIG. 4E—The SLi that show a significant predictive signal when applied to predict clinical drug response in breast cancer. Edge width denotes the significance of the SLi according to the clinical data. SLi edges that are also supported by in-vitro drug response data are for example KDELR3, ARPC1B, CEP55, HSCBP1, TMSB10, ATP5J2, KIFC3, YIS1A, SERPINH1, TNFRSF12, MPZL2, LCN2, NFIB, LCN2, BACE2, PSMB10³⁹⁻⁴¹.

FIGS. 5A-5E. The clinical-SL-network predicts the response of NSCLC patients to erlotinib. FIG. 5A-5B—The predicted response to erlotinib (y-axis) vs. months-to-progression (x-axis) observed in the NSCLC patients treated with erlotinib (FIG. 5A), and sorafenib (FIG. 5B). FIG. 5C—The predicted response to erlotinib (y-axis) in five groups of NSCLC patients treated with erlotinib, divided uniformly according to their observed months-to-progression. FIG. 5D—The gene expression of the EGFR SL-partners in patients treated with erlotinib, ordered according to their months-to-progression (on-top). As predicted, patients with many underexpressed EGFR SL-partners progressed slower. FIG. 5E—The predicted SL-partners of EGFR that have been validated based on the clinical drug response to erlotinib. The edge width denotes the significance of the SLi according to the clinical data. Some SLi are also supported by in-vitro drug response data.

FIGS. 6A-6F. Characterizing and utilizing the miRNA-SL-network. FIGS. 6A and 6E—The weighted degree of miRNAs in the SL-network as a function of their involvement in cancer according to the literature: OncomiRDB64 (right) and miRCancer⁶⁵ (left). FIGS. 6B, 6F, and 6G—The survival KM-plots of patients that were predicted as responders (upper line in all plots) and non-responders (lower line in all plots) to the treatment they received, shown when considering all drugs in The Cancer Genome Atlas (TCGA) and individually, for two frequently administered drugs. FIG. 6C—The KM-plot depicting the survival rates of tamoxifen treated breast cancer patients that were classified as responders (upper line in all plots) and non-responders (lower line in all plots) based on the expression of the miRNA-SL-partners of ESR1/2 in each sample. FIGS. 6B-C—In parenthesis next to the name of each group are the number of patients and the number and fraction of deaths in that group. FIG. 6D—The SLi partners (mir-n) of drug targets (ESR1, MAPT, TOP2A) that are strongly supported by clinical drug response data along with the genes targeted by the miRNAs according to both ENCODE58 and miRTarBase⁵⁹.

FIGS. 7A-7D. Regulatory and asymmetric SLi. Upon the loss of a specific tumor suppressor A, the downstream oncogenic pathway it inhibits is likely to become overactive. This may occur due to impaired A-dependent regulatory mechanisms, or due to sequential beneficial genomic alterations. Activation (indicated by bold arrow) of this oncogenic pathway diminishes selection pressure to maintain collateral pathways which are gradually inactivated due to genetic or epigenetic changes, leading to oncogene addiction.

FIGS. 8A-8D. The concepts of clinically-relevant and cumulative SLi represented by theoretical fitness curves. FIG. 8A—Classical SLi: Given the SLi between genes A and B, complete neutralization of B, genetically or pharmacologically, would have no effect on normal cells and will be lethal to A-deficient cells. FIG. 8B—Classical and clinically-relevant SLi: To make the SLi between A and B of clinical relevance it is of essence that even partial inhibition of B in A-deficient cancer cells will cause death or a significant selective disadvantage. FIG. 8C—Clinically-relevant SLi: B-inhibitors might display a significant therapeutic index even if B is required also when A is active. If gene B has additional cumulative SL-partners, as gene C, then the therapeutic window can be further extended in cancer cells deficient for both A and C. FIG. 8D—Vann diagram demonstrating the theoretical intersection between SLi according to the classical definition (FIGS. 8A, 8B), and according to the less stringent and therapeutic-oriented definition (FIG. 8C).

FIG. 9. Gene Ontology enrichment. Overrepresented GO terms among the 500 genes with the highest weighted (A) in-degree, (B) out-degree, and (C) out-degree when considering (A)-(B) all SLi in the network or (C) only SLi of the form A→T such that T is an oncology drug target.

FIGS. 10A-10E. The SL-network predicts treatment outcomes in cancer patients. The survival Kaplan Meier (KM)-plots of patients that were predicted as responders (upper line in all plots) and non-responders (lower line in all plots) to the treatment they received, shown when considering all drugs in the dataset and individually for a few frequently administered drugs. In parenthesis next to the name of each group are the number of patients and the number and percentage of deaths in that group.

### DETAILED DESCRIPTION OF THE INVENTION

According to some embodiments the present invention provides systems and methods for identification and creation of Synthetic Lethal interactions (SLi) and networks and uses thereof to uncover therapeutic possibilities and drug response, for example, in treating cancer in subjects in need thereof.

According to some embodiments, a new pipeline for identifying cancer Synthetic Lethal interactions (SLi), is provided. This new method (pipeline) is termed herein, SL-Identification based on Clinical Knowledge (SLICK). Extending upon previously described method, DAISY, SLICK goes beyond molecular omics (genomics, transcriptomics, proteomics or metabolomics) data and analyzes also clinical survival and drug response data to identify SLi. SLICK was applied in systems and methods as disclosed herein, to generate a clinically-derived pan-cancer SL-network. The network accurately predicts the results obtained in several gene essentiality and drugs response screens³⁷⁻⁴¹. As demonstrated hereinbelow, novel predictions obtained by the network were experimentally validated, involving drug repurposing, context-specific drug efficacy, and synergistic drug combinations. It is demonstrated herein for the first time that an SL-network can successfully predict treatment outcomes in cancer patients.

According to some embodiments, the SLICK method and system provided in the present invention markedly extend the ability to harness synthetic lethality to cancer and advance precision medicine.

### DEFINITIONS

Synthetic lethal interactions (SLi) describe the relationship between two genes whereby an individual inactivation of either gene results in a viable phenotype, while their combined inactivation is lethal

Synthetic lethality (SL) occurs when a perturbation of two nonessential genes is lethal.

Synthetic Dosage Lethality (SDL) denotes an interaction between two genes in which the over-activity of one gene renders the other gene essential.

SLi-based treatment refer to treatment of a condition (such as, cancer) with known, repurposed or newly identified, agents or combination of agents capable of targeting at least one gene present in SLi network identified according to the methods and systems of the present invention.

Somatic copy Number of Alterations (SCNA) refer to somatic changes to chromosome structure that result in gain or loss in copies of sections of DNA, and are prevalent in many types of cancer.

Messenger RNA (mRNA) is a large family of RNA molecules that convey genetic information from DNA to the ribosome, where they specify the amino acid sequence of the protein products of gene expression. mRNA genetic information is in the sequence of nucleotides, which are arranged into codons consisting of three bases each.

A small hairpin RNA or short hairpin RNA (shRNA) is a sequence of RNA that makes a tight hairpin turn that can be used to silence target gene expression via RNA interference (RNAi). Expression of shRNA in cells is typically accomplished by delivery of plasmids or through viral or bacterial vectors.

Small interfering RNA (siRNA), sometimes known as short interfering RNA or silencing RNA, is a class of double-stranded RNA molecules, 20-25 base pairs in length. siRNA plays many roles, but it is most notable in the RNA interference (RNAi) pathway, where it interferes with the expression of specific genes with complementary nucleotide sequences. siRNA functions by causing mRNA to be broken down after transcription, resulting in no translation.

The terms “cancer” and “cancerous” refer to or describe the physiological condition in mammals that is typically characterized by unregulated cell growth. Examples of cancer include but are not limited to, carcinoma, lymphoma, blastoma, sarcoma, and leukemia. More particular examples of such cancers include squamous cell cancer, lung cancer (including small-cell lung cancer, non-small-cell lung cancer, adenocarcinoma of the lung, and squamous carcinoma of the lung), cancer of the peritoneum, hepatocellular cancer, gastric or stomach cancer (including gastrointestinal cancer), pancreatic cancer, glioblastoma, cervical cancer, ovarian cancer, liver cancer, bladder cancer, hepatoma, breast cancer, colon cancer, colorectal cancer, endometrial or uterine carcinoma, salivary gland carcinoma, kidney or renal cancer, liver cancer, prostate cancer, vulval cancer, thyroid cancer, hepatic carcinoma and various types of head and neck cancer, as well as B-cell lymphoma (including low grade/follicular non-Hodgkin's lymphoma (NHL); small lymphocytic NHL; intermediate grade/follicular NHL; intermediate grade diffuse NHL; high grade immunoblastic NHL; high grade lymphoblastic NHL; high-grade small non-cleaved cell NHL; bulky disease NHL; mantle cell lymphoma; AIDS-related lymphoma; and Waldenstrom's Macroglobulinemia); chronic lymphocytic leukemia (CLL); acute lymphoblastic leukemia (ALL); Hairy cell leukemia; chronic myeloblastic leukemia; and post-transplant lymphoproliferative disorder (PTLD), as well as abnormal vascular proliferation associated with phakomatoses, edema (such as that associated with brain tumors), and Meigs' syndrome.

The terms “anti-neoplastic” and “anti-cancer” may interchangeably be used. The term “anti-neoplastic composition” refers to a composition useful in treating cancer comprising at least one active therapeutic agent capable of inhibiting or preventing tumor growth or function or metastasis, and/or causing destruction of tumor cells. Therapeutic agents suitable in an anti-neoplastic composition for treating cancer include, but not limited to, chemotherapeutic agents, radioactive isotopes, toxins, cytokines such as interferons, and antagonistic agents targeting cytokines, cytokine receptors or antigens associated with tumor cells. For example, therapeutic agents useful in the present invention can be antibodies such as anti-HER2 antibody and anti-CD20 antibody, or small molecule tyrosine kinase inhibitors such as VEGF receptor inhibitors and EGF receptor inhibitors. Preferably the therapeutic agent is a chemotherapeutic agent.

The terms “chemotherapeutic agent”, “anti-cancer drug” and “anti-cancer agent” may interchangeably be used. The terms relate to a chemical compound useful in the treatment of cancer. Examples of chemotherapeutic agents include alkylating agents such as thiotepa and cyclosphosphamide; alkyl sulfonates such as busulfan, improsulfan and piposulfan; aziridines such as benzodopa, carboquone, meturedopa, and uredopa; ethylenimines and methylamelamines including altretamine, triethylenemelamine, trietylenephosphoramide, triethiylenethiophosphoramide and trimethylolomelamine; acetogenins (especially bullatacin and bullatacinone); a camptothecin (including the synthetic analogue topotecan); bryostatin; callystatin; CC-1065 (including its adozelesin, carzelesin and bizelesin synthetic analogues); cryptophycins (particularly cryptophycin 1 and cryptophycin 8); dolastatin; duocarmycin (including the synthetic analogues, KW-2189 and CB1-TM1); eleutherobin; pancratistatin; a sarcodictyin; spongistatin; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, and ranimnustine; antibiotics such as the enediyne antibiotics (e. g., calicheamicin, especially calicheamicin gamma1I and calicheamicin omegaI1; dynemicin, including dynemicin A; bisphosphonates, such as clodronate; an esperamicin; as well as neocarzinostatin chromophore and related chromoprotein enediyne antibiotic chromophores), aclacinomycins, actinomycin, authramycin, azaserine, bleomycins, cactinomycin, carabicin, carminomycin, carzinophilin, chromomycins, dactinomycin, daunorubicin, detorubicin, 6-diazo-5-oxo-L-norleucine, doxorubicin (including morpholino-doxorubicin, cyanomorpholino-doxorubicin, 2-pyrrolino-doxorubicin and deoxydoxorubicin), epirubicin, esorubicin, idarubicin, marcellomycin, mitomycins such as mitomycin C, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; eniluracil; amsacrine; bestrabucil; bisantrene; edatraxate; defofamine; demecolcine; diaziquone; elfornithine; elliptinium acetate; an epothilone; etoglucid; gallium nitrate; hydroxyurea; lentinan; lonidainine; maytansinoids such as maytansine and ansamitocins; mitoguazone; mitoxantrone; mopidanmol; nitraerine; pentostatin; phenamet; pirarubicin; losoxantrone; podophyllinic acid; 2-ethylhydrazide; procarbazine; PSK® polysaccharide complex (JHS Natural Products, Eugene, Oreg.); razoxane; rhizoxin; sizofiran; spirogermanium; tenuazonic acid; triaziquone; 2,2′,2″-trichlorotriethylamine; trichothecenes (especially T-2 toxin, verracurin A, roridin A and anguidine); urethan; vindesine; dacarbazine; mannomustine; mitobronitol; mitolactol; pipobroman; gacytosine; arabinoside (“Ara-C”); cyclophosphamide; thiotepa; taxoids, e.g., paclitaxel and doxetaxel; chlorambucil; gemcitabine; 6-thioguanine; mercaptopurine; methotrexate; platinum coordination complexes such as cisplatin, oxaliplatin and carboplatin; vinblastine; platinum; etoposide (VP-16); ifosfamide; mitoxantrone; vincristine; vinorelbine; novantrone; teniposide; edatrexate; daunomycin; aminopterin; xeloda; ibandronate; irinotecan (e.g., CPT-11); topoisomerase inhibitor RFS 2000; difluorometlhylornithine (DMFO); retinoids such as retinoic acid; capecitabine; and pharmaceutically acceptable salts, acids or derivatives of any of the above.

Also included in this definition are anti-hormonal agents that act to regulate or inhibit hormone action on tumors such as anti-estrogens and selective estrogen receptor modulators (SERMs), including, for example, tamoxifen, raloxifene, droloxifene, 4-hydroxytamoxifen, trioxifene, keoxifene, LY117018, onapristone, and toremifene; aromatase inhibitors that inhibit the enzyme aromatase, which regulates estrogen production in the adrenal glands, such as, for example, 4(5)-imidazoles, aminoglutethimide, megestrol acetate, Aexemestane, formestanie, fadrozole, vorozole, letrozole, and Aanastrozole; and anti-androgens such as flutamide, nilutamide, bicalutamide, leuprolide, and goserelin; as well as troxacitabine (a 1,3-dioxolane nucleoside cytosine analog); antisense oligonucleotides, particularly those which inhibit expression of genes in signaling pathways implicated in aberrant cell proliferation, such as, for example, PKC-alpha, Raf and H-Ras; ribozymes such as a VEGF expression inhibitor (e.g., ANGIOZYME® ribozyme) and a HER2 expression inhibitor; vaccines such as gene therapy DNA-based vaccines, for example, ALLOVECTIN® vaccine, LEUVECTIN® vaccine, and VAXID® vaccine; PROLEUKIN® rIL-2; LURTOTECAN® topoisomerase 1 inhibitor; ABARELIX® rmRH; and pharmaceutically acceptable salts, acids or derivatives of any of the above.

The term “repurposing” is directed to repurposing known active ingredients which are used for treating a first condition in the therapy of a different condition, such as, cancer therapy.

The terms “subject” and “patient” may interchangeably be used. The terms relate to a subject afflicted with a condition. In some exemplary embodiments, the condition is cancer.

The term “DAISY” is directed to DAta-mining SYnthetic-lethality identification pipeline (DAISY), which is used for identifying cancer SLi.

### The Computational Aspect

In some embodiments, the methods disclosed herein may be implemented by computational means, utilizing one or more computing units. In computer science, a graph is an abstract data type used for implementing the graph concept from mathematics. A graph may be implemented in a multiplicity of ways, using various data structures, data structure collections, linking mechanisms such as but not limited to pointers, or the like. A graph generally comprises nodes (also referred to as vertices) and edges connecting two nodes. In many cases, each node represents an object and each edge represents a connection between object. In some cases, each edge may be associated with one or more properties, such as an identifier or quantifier associated with the connection between the objects, such as weight, significance or other properties. Edges may be directional or bidirectional.

According to some embodiments, the system of the present invention may be used to execute the computing methods (i.e. SLICK) to generate the results (SLi networks and data derived therefrom). In some embodiments, the system generally comprise a computing platform, comprising one or more processors, any of which may be any processing circuitry, such as Central Processing Unit (CPU), a microprocessor, an electronic circuit, an Integrated Circuit (IC) or the like. The processor 204 can be implemented as hardware or configurable hardware such as field programmable gate array (FPGA) or application specific integrated circuit (ASIC). In other alternatives, the processor can be implemented as firmware written for or ported to a specific processor such as digital signal processor (DSP) or microcontrollers. The processor may be used for performing mathematical, logical or any other instructions required by computing platform or any of it subcomponents. In some embodiments, the computing platform may include an input/output device, such as a keyboard, a mouse, a touch screen, a display, or any other device used for receiving data or commands from a user, or displaying options or output to the user. In some exemplary embodiments, the computing platform may comprise or be associated with one or more storage devices. The storage device may be non-transitory (non-volatile) or transitory (volatile). For example, storage device can be a Flash disk, a Random Access Memory (RAM), a memory chip, an optical storage device such as a CD, a DVD, or a laser disk; a magnetic storage device such as a tape, a hard disk, storage area network (SAN), a network attached storage (NAS), or others; a semiconductor storage device such as Flash device, memory stick, or the like. The storage device may contain user interface component for receiving input or providing output to and from a server or a user.

In some embodiments, the storage device may further contain graph implementation component for performing calculations for creating and manipulating graphs, for example intersecting graphs. Creating the graph may use calculations involving data from the available results. The storage device may further comprise graph analysis component for analyzing the constructed graphs, and drawing conclusions, such as for identifying effective treatment for a patient, assessing effectiveness of a treatment, providing prognosis for a patient, determining drug response, determining drug efficacy, and the like. In some embodiments, storage device 220 may also store data such as, databases information, clinical data and results.

According to some embodiments, the methods of identifying SLi interactions, generating networks, and determining related therapeutic effects (such as SLICK), may be utilized using a direct data-driven computational system.

### SL-Identification Based on Clinical Knowledge (SLICK)

According to some embodiments, SLICK can be used to identify SLi that occur in-vivo and are likely to have a therapeutic value. SLICK is based on the observation that cancer cells are shaped by clonal selection. Following this rationale, SLICK identifies SLi by detecting underrepresented genomic and transcriptomic patterns that are likely to be deleterious to the tumor, namely, patterns which are selected against in the cancer cell population and are strongly associated with patient survival.

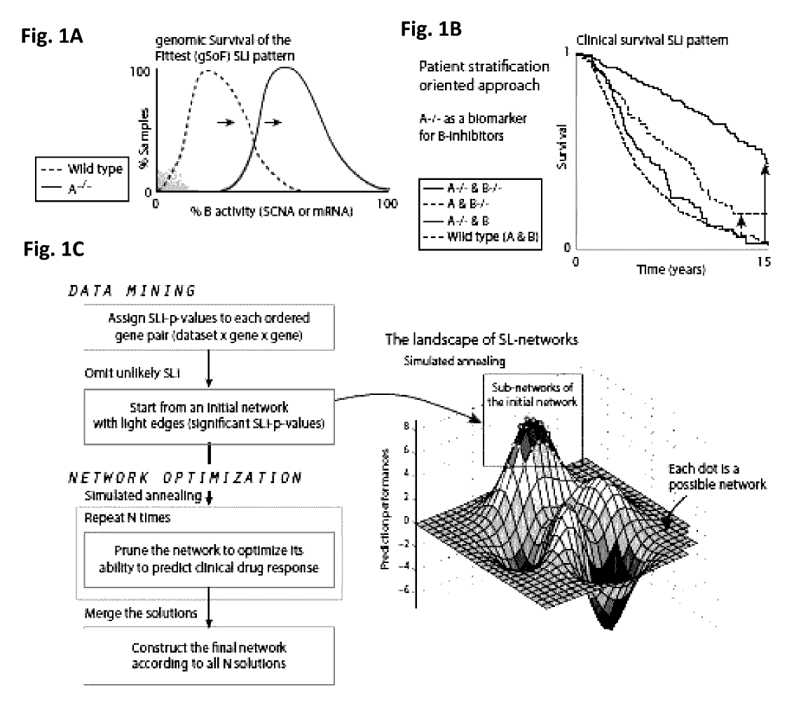

In some embodiments, the SLICK method includes two main steps. In some embodiments, the steps may be executed by the computation system. In some embodiments, in the first step—the data-mining step—SLICK performs integrative genome-wide analyses of molecular omics data and patient survival data. It examines for each pair of genes whether it exhibits the molecular and clinical patterns characteristic of SL-partners, and generates an initial draft SL-network (demonstrated in FIG. 1A-1B). In the second step—the network optimization step—SLICK eliminates false positive predictions that may have emerged in the first step by optimizing the network ability to predict clinical drug response (demonstrated in FIG. 1C).

According to some embodiments, in the first, data-mining step, SLICK computes for each pair of genes a set of SLi-p-values that denote its likelihood of being SL. This is done by employing three types of data-driven inference procedures:

I. genomic Survival of the Fittest (gSoF): A gSoF-SLi-pattern between two genes (A and B) denotes that upon the loss of gene A the Somatic Copy Number Alterations (SCNA) and mRNA levels of gene B are significantly increased (FIG. 1A). Such patterns can arise due to selection pressures that eliminate cancer cells with inactive SL-paired genes or due to compensatory mechanisms that upregulate the SL-partners of inactive genes. Similarly to DAISY, SLICK analyzes thousands of cancer SCNA and mRNA profiles, and denotes for each gene pair if it manifests the gSoF-SLi-pattern. One caveat of the gSoF approach is that gSoF patterns may also arise due to the redundancy of genetic alterations^{35,36,}. For example, the loss of a tumor suppressor may alleviate the selection pressure to lose another tumor suppressor. Therefore, although such pairs of tumor suppressors are not SL, they may display a strong gSoF-SL-pattern. Improving upon DAISY, SLICK eliminates such false positive predictions by employing clinical survival analyses and network pruning as describe hereafter.
II. Clinical survival analyses: Patients with aggressive and resistant tumors are likely to have lower survival rates, and vice versa. The survival rates of cancer patients hence provide a rough approximation to the fitness of their tumors. In light of this observation SLICK identifies SLi by analyzing the genomic and molecular profiles of tumors along with the survival rates of the patients bearing them. It identifies genes A and B as candidate SL partners if the inactivity of gene B is associated with improved prognosis specifically in the subpopulation of patients with A-deficient tumors (FIG. 1B). This implies that A-deficiency induced the essentiality of gene B and reveals its oncogenic potential. Moreover, it indicates that A-deficiency may be a marker for response to a treatment with B-inhibitors. Further explanations and clarifications concerning these analyses are provided in the experimental section.
III. Correlated expression: The expression of SL-partners may tend to be positively correlated, as SL-partners often participate in related biological processes which are upregulated under the same conditions³⁰. On the other hand, the expression of SL-partners may be negatively correlated due to the gSoF-SLi-pattern. Additionally, in regulatory SLi the tumor suppressor is a negative regulator of the oncogene and therefore their expression is more likely to be inversely correlated. SLICK hence considers both positive and negative correlations as potential characteristics of SLi.

According to some embodiments, the above mentioned concepts were translated to six statistical procedures (approaches). In difference from DAISY, each inference procedure is applied when considering the data collected for a specific cancer type in a specific study, resulting in the assignment of a few dozen SLi-p-values to each ordered pair of genes. The initial draft network then consists of gene pairs with an array of overall statistically significant SLi-p-values.

According to some embodiments, in the second, optimization step, SLICK prunes the initial draft network by optimizing its ability to predict clinical drug response. It further exploits the value embedded in the SLi-p-values assigned to each individual candidate interaction to reduce the search space and regularize the fit to the training clinical drug response data. More specifically, in some embodiments, SLICK employs an optimization heuristic called Simulated Annealing (SA) to examine different ways by which the SLi-p-values can be combined to discriminate between true and false predictions of SLi. In some embodiments, it then selects the setting that obtains the most predictive and thus clinically-relevant network according to the clinical drug response (training) data. In some embodiments, SLICK performs this process 1,000 times using different subsets of the training data, resulting in 1,000 networks or solutions. In some embodiments, it may then aggregates the different solutions into a weighted network such that the weight or strength of a given SLi is the fraction of solutions in which it appeared.

### Predicting Clinical Drug Response Using SLICK

In some embodiments, to assess the ability of an SL-network to predict clinical drug response the network is first integrated with the gene expression profiles of the patients' tumors. The response of a patient to a specific drug may be predicted in an unsupervised, straight forward manner as proportional to the number of underexpressed SL-partners the specific drug target(s) has in the patient's tumor. Based on the latter, the patients may be classified as responders and non-responders to the specific treatment they received, and a logrank p-value is computed to examine whether the responders outlived the non-responders. In some embodiments, a control logrank p-value is additionally computed using randomly shuffled drug-patient mappings. The ratio between these two p-values is the objective function guiding the simulated annealing (SA) optimization process. It denotes the ability of the SL-network to specifically predict drug response while controlling for drug-independent patient survival rates.

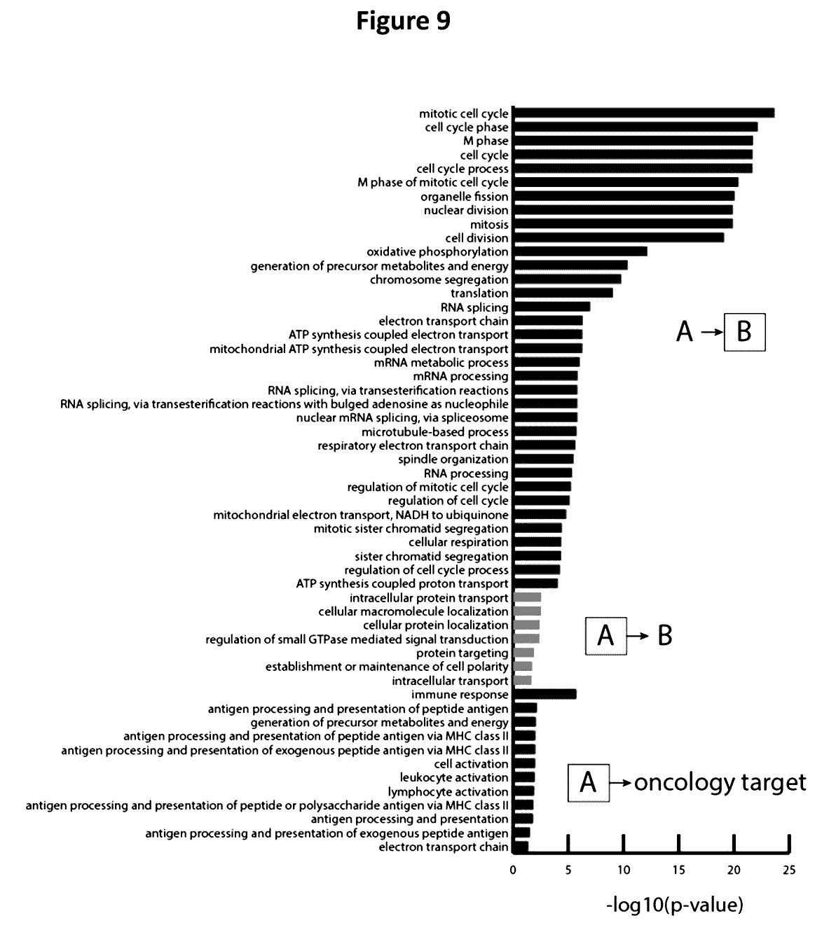

In some exemplary embodiments, SLICK was applied based on the Somatic Copy Number Alterations (SCNA), gene expression, somatic mutation profiles, treatment information, and survival data of overall 5,417 clinical samples. The resulting pan-cancer clinical-SL-network, consists of 13,906 genes and 635,447 SLi—the majority of which (85.8%) are asymmetric (as presented in FIG. 9).

According to some embodiments, tumor fitness is determined by a complex set of different selective pressures—the need to evade the immune system, harness non-cancerous cells, promote angiogenesis, metastasize, and more. Thus, SLi that occur in-vitro may not occur in-vivo, and vice versa. Bearing this in mind, SLICK is designed, in some embodiments, to detect in-vivo SLi. Although it optimizes the network to predict clinical drug response, its network successfully predicts in-vitro susceptibilities as well. The SL-network generated by SLICK could therefore provide, in some embodiments, a research platform and help bridge the gap between experimental models and the clinic.

According to some embodiments, SLICK performances may be further improved with the rapid accumulation of cancer omics data—especially clinical survival and drug response data that are currently limited. As SLICK relies on the assessment of gene activity, it could be enhanced by analyzing additional data types such as single-cell gene expression data, proteomics, protein modifications, and epigenetic alterations. In some embodiments, the methods and systems disclosed herein may further be utilized for the identification of SLi involving long-non-coding RNA and other non-coding regulatory genomic elements as enhancers and silencers. In some embodiments, the methods and systems disclosed herein may further be employed to identify cancer type-specific SL-networks. In some embodiments, the examples herein focused on pan-cancer SLi that are shared among different cancer types. Notably, although oral and pancreatic cancers where underrepresented in the network construction data the network predictions were successfully experimentally validated in these cancer types.

According to some embodiments, as further demonstrated herein, SLICK can be applied to identify promising candidates for drug repurposing and drug combinations in a manner that enables one to detect also a subpopulation of responsive patients. The treatment efficacy and selectivity could potentially be further enhanced by identifying drug combinations that are especially synergistic in the tumor. Additionally, it is shown for several widely-used drugs that the state of their predicted SL-partners in the tumors is predictive of clinical drug response of the patients. These findings indicate that cancer drugs in use today may be exploiting SLi, even though their action has not been explicitly attributed to such mechanisms.

According to some embodiments, SLICK may be used to predict clinical drug response in a manner that is fundamentally different and complementary to most current cancer precision medicine approaches. The SLICK approach has three important advantageous distinguishing properties: First, its predictions are based on a genome-scale view of SLi. Such a systematic approach could potentially be more effective when treating heterogeneous tumors and help avoid the emergence of drug resistance. Second, unlike specific biomarkers and genetic signatures that have been developed to predict the response to specific treatments, SLICK is a generic clinical drug response prediction platform. Third, unlike previous attempts to realize the clinical potential of SLi, SLICK identifies and utilizes cumulative SLi that could broaden the ability to detect responsive patients. In some embodiments, SLICK strategy could therefore be promising in particular for patients with tumors that do not bear specific oncogenic mutations.

According to some embodiment, as demonstrated and exemplified herein, by the results presented, SLICK obtains accurate and valuable predictions across numerous and varied clinical datasets and experimental systems. All in all, SLICK lays a basis for advancing the understanding of drug response in cancer and addressing key translational challenges.

Pharmaceutical compositions according to the present invention may be administered by any suitable means, such as orally, topically, intranasally, subcutaneously, intramuscularly, intravenously, intra-arterially, intraarticulary, intralesionally or parenterally.

In some embodiments, it will be apparent to those of ordinary skill in the art that the therapeutically effective amount of a molecule according to the present invention will depend, inter alia upon the administration schedule, the unit dose of molecule administered, whether the molecule is administered in combination with other therapeutic agents, the immune status and health of the patient, the therapeutic activity of the molecule administered and the judgment of the treating physician.

### Underexpression Definition

Two definitions were used to identify underexpressed genes. A gene is denoted as underexpressed or strongly underexpressed in a certain sample if its expression in this sample is below the median or 10^{th} percentile of its expression across all the samples in the pertaining dataset.

In some embodiments, SLICK method and system disclosed herein consists of two main steps (FIGS. 1A-1C). In the first step, termed the data mining step, SLICK performs integrative genome-wide analyses of molecular omics data and patient survival data. It assigns each pair of genes a set of SLi-p-values that denote whether it exhibits the molecular and clinical patterns characteristic of SL-partners (FIGS. 1A-1B). Next, SLICK constructs an initial draft network that consists of candidate SLi. In the second step, termed the network optimization step, SLICK prunes the initial network to optimize it according to a given objective (FIG. 1C). Various objectives can be used, depending on the type of SLi one aims to identify. The objective used in the current exemplified implementation is to predict drug response in cancer patients, aiming to identify SLi that occur in the clinic and have a therapeutic value.

In some embodiments, in the first, data-mining step, SLICK computes for each ordered pair of genes a set of SLi-p-values. As SLi are asymmetric, the SLi in which the inactivity of gene A induces the essentiality of gene B is denoted by

$$A {->}^{SL} B .$$

The data-driven inference procedures that compute the SLi-p-values for a specific pair of genes, denoted throughout as A and B is detailed below.

According to some embodiments, one of the characteristics of SLi is the gSoF-SLi-pattern. In some embodiments, this pattern denotes that upon the loss of gene A, the SCNA and mRNA levels of gene B are significantly increased (FIG. 1A). To examine whether A and B show the gSoF-SLi-pattern, two complementary gSoF analyses are performed. SCNA-based gSoF analyses examine whether the SCNA levels of gene B are significantly higher in samples with inactive A compared to samples with active A. These analyses examine whether A-inactivity results in a purifying selection that eliminates cells with a low copy-number of gene B. mRNA-based gSoF analyses examine whether the mRNA levels of gene B are significantly higher in A-deficient samples compared to other samples. These analyses examine whether the inactivity of gene A induces compensating mechanisms that upregulate gene B. Such mechanisms may or may not be mediated by copy-number alterations. The mRNA-based analyses are especially important as SCNA are not necessarily correlated to gene expression. Both gSoF analyses are conducted by employing one-sided Wilcoxon ranksum tests, and defining gene A as inactive in a sample if it is strongly underexpressed and its SCNA is below −0.3.

In some embodiments, clinical SLi analyses are performed specifically to identify SLi predictive of clinical drug response. First, explained is the rationale guiding these analyses, as it is different than the conventional view of SLi. Then described is how the clinical SLi analyses are implemented. According to the classical definition, A and B are SL-partners if and only if the double mutant for both A and B has a lower fitness compared to its expected fitness²⁸. The expected fitness is estimated based on the fitness of the single A and B mutants. The genetic interaction between two genes is then conventionally quantified by

ε_{AB}=ƒ_{Ā̂B̅}−ƒ_{Ā̂B}ƒ_{ÂB̅} (1)

where ƒ_{Ā̂B} and ƒ_{ÂB̅} denote the fitness of the single A and B mutants, respectively, and ƒ_{Ā̂B̅} denotes the fitness of the double mutant. A negative ε_{AB} value indicates that A and B are SL-partners, namely, that the fitness of the double mutant is lower than expected. The magnitude of ε_{AB}, meaning |ε_{AB}|, is the estimated strength of the interaction.

If it is assumed that the survival rates of cancer patients are negatively correlated to the fitness of their tumors these definitions can be translated to the clinical setting. Let S_{Ā̂B} and S_{ÂB̅} denote the subpopulations of patients with A-deficient and B-deficient tumors, respectively. Additionally, denote by S_{Ā̂B̅} and S_{ÂB} the subpopulations of patients bearing tumors deficient for both A and B, or tumors with functionally active A and B, respectively. According to the ε_{AB} measure it should first be examined whether S_{Ā̂B}, S_{ÂB̅}, and S_{Ā̂B̅} patients outlived S_{ÂB} patients, obtaining three logrank p-values, denoted as P_{Ā̂B}, P_{ÂB̅}, and P_{Ā̂B̅}, respectively. This is equivalent to normalizing the fitness of a mutant strains by the fitness of the wild type strain, represented here by S_{ÂB}. Then it should be concluded that A and B are more likely to be SL-partners if P_{Ā B̅} is significantly smaller than what can be expected based on P_{ÂB̅} and P_{Ā̂B}. Indeed, such a phenomenon may indicate that treating S_{ÂB} patients with both A-inhibitors and B-inhibitors is likely to have a synergistic effect. However, this type of analysis is suboptimal if one aims to utilize the network to discriminate between responders and non-responders to a single drug. In some embodiments, an SL-network that can correctly discriminate between responders and non-responders to a specific treatment can be applied to tackle a variety of translational objectives. For example, it can identify novel drugs and novel targets for cancer treatment by assessing the size of the responsive subpopulation of patients.

According to some embodiments, the aim is therefore to identify

$$A {->}^{SL} B$$

that will indicate A-deficiency is a marker for response to B-inhibitors. It is hence necessary to estimate what will be the effect of B-inhibition in S_{Ā̂B} patients compared to S_{ÂB} patients. SLICK estimates the effect of B-inhibition in S_{Ā̂B} patients by examining if S_{Ā̂B̅} outlived S_{Ā̂B} patients, resulting in a logrank p-value denoted as P_{Ā}. Similarly, SLICK examines if S_{Ā̂B̅} patients outlived S_{ÂB} patients, and computes another logrank p-value, denoted as P_{A}. The final normalized p-value according to this inference procedure is then

$${P}_{clinic} = \frac{{P}_{\overline{A}}}{{P}_{A}} .$$

According to some embodiments, the method and system may employ two definitions—strict and permissive—to classify the patients into these four subpopulations, namely, S_{ÂB}, S_{Ā̂B}, S_{ÂB̅}, and S_{Ā̂B̅}. It then computes two P_{clinic} p-values, one according to each definition. Let S denote all the clinical samples in the dataset. We define S_{Ā}=S_{Ā̂B} ∪S_{Ā̂B̅} and S_{A}=S\S_{Ā}. The strict definition classifies samples as S_{Ā} if they strongly underexpress A or if they have a deleterious mutation in A. The latter refers to nonsense and frame-shift mutations. The second, more permissive definition, classifies samples as S_{Ā} if they underexpress A. According to either the strict or permissive definition of S_{Ā} the samples are further classified according to their expression of gene B, as follows. Let M_{BεS}_{_{A}} and M_{BεS}_{_{Ā}} be the median expression of gene B in S_{A} and S_{Ā}, respectively. A sample is classified as S_{Ā̂B} or S_{Ā̂B̅} if it is classified as S_{Ā} and expressed B at a level above or below M_{BεS}_{_{Ā}}, respectively. Similarly, a sample is classified as S_{ÂB} or S_{ÂB̅} if it is classified as S_{A} and expressed B at a level above or below M_{BεS}_{_{A}}, respectively. Of note, in the permissive version the size of all four groups is identical: |S_{ÂB}|=|S_{Ā̂B}|=|S_{ÂB̅}|=|S_{Ā̂B̅}|. Hence, P_{Ā} and P_{A} are comparable, meaning that the ratio between them is not skewed or biased toward high or low values due to differences in sample size. In the strict version |S_{Ā̂B}|+|S_{Ā̂B̅}|<|S_{ÂB}|+|S_{ÂB̅} | and hence the resulting normalized p-value is more likely to be skewed toward larger values, perhaps resulting in false negative predictions. However, this can be corrected by the SA process that can define higher thresholds for the strict-clinical SLi-p-values, as explained in the section below.

In some embodiments, the SLICK methods and system may further compute the Spearman correlation between the mRNA levels of each pair of genes across the cancer samples of the pertaining dataset. This analysis results in two one-sided p-values, one denotes whether the two genes are negatively correlated, and the other denotes whether the two genes are positively correlated. These two p-values are then used in the SA process such that each has a different threshold associated with it.

According to some embodiments, each of the six inference procedure described above may be applied when considering the data collected for a specific cancer type in a specific study. Let nᵢ be the number of datasets analyzed by inference procedure i, then procedure i generates nᵢ p-values for each ordered pair of genes. Overall there are m=Σᵢ₌₁⁶ nᵢ SLi-p-values for each ordered gene pair. These m p-values are aggregated into a single p-value via a Fisher's combined probability test and corrected for multiple hypotheses testing via Bonferroni correction. Gene pairs whose combined corrected p-value is below 0.05 and have at least five p-values <1e-3 form the initial SL-network.

According to some embodiments, in the second, network optimization step SLICK prunes the initial network to optimize it according to a specific objective function. An SL-network can be defined as G=(V,E), where V denotes the network nodes, such that each node represents a gene, and E denotes network edges, such that each edge corresponds to an SLi: E={(A,B)|A,BεV and

$$A {\to }^{SL} B \} .$$

Given an SL-network the objective function ƒ returns a score that quantifies the network performances in a certain task, meaning, ƒ: (V,E)→ [symbol] . SLICK operates a heuristic optimization algorithm called SA that aims to find a subnetwork of the initial draft network (V_{I},E_{I}) with the maximal ƒ(V,E) score, meaning

argmax_{(V,E)|V⊂V}_{_{I}}_{,E⊂E}_{_{I}}{ƒ(V,E)} (2)

Various objective functions can be use, depending on the type of SLi one aims to identify. The objective function used in the current implementation is described in the next subsection.
The pruning process accounts for the SLi-p-values to reduce the search space and regularize the fit to the data used by the objective function. In our case, the latter is the clinical drug response data, which is at times noisy and incomplete. The initial network is hence defined by G_{I}=(V_{I}, E_{I}, W₁, . . . Wₘ), where V_{I} and E_{I} are defined as explained above and Wᵢ denotes the SLi-p-values, or weights, which were assigned to the network edges according to one of the inference procedures when applied on one of the datasets. Based on the initial network two cutoffs are defined for each Wᵢ: a stringent cutoff W_{i,s}=min(1e-3, 5^{th} percentile of Wᵢ), and a permissive cutoff W_{i,p}=min(0.05, 20^{th} percentile of Wᵢ).
The number of possible subnetworks is exponential

|{(V,E)|V⊂V_{I},E⊂E_{I}}|=2^{|E}^{_{I}}^{|} (3)

SLICK transform this large space of possible subnetworks to the space of valid pruning vectors: Pε{0,1,2}^{m}. Each pruning vector defines a subnetwork. If Pᵢ=0, Wᵢ is disregarded. If Pᵢ=1, Wᵢ is accounted for in a permissive manner, meaning that an SLi will be supported by Wᵢ if W_{i,(A,B)}<W_{i,p}. If Pᵢ=2, Wᵢ is accounted for in a stringent manner, meaning that an SLi will be supported by Wᵢ if W_{i,(A,B)}<W_{i,s}. The resulting subnetwork then consists of interactions which are supported by at least C₁ p-values. In the current implementation C₁=5. According to the above we can define a mapping g that given a pruning vector returns the subnetwork it yields

g:{0,1,2}^{m}→{(V,E)|V⊂V_{I},E⊂E_{I}} (4)

The optimization problem described in (2) is hence reduced to

argmax_{Pε{0,1,2}}_{^{m}}{ƒ(g(P))} (5)

The SA process starts from a random state within the search space, that is, a subnetwork (Vₜ,Eₜ) defined based on a randomly selected pruning vector. It examines the performances of the network based on the objective function by computing ƒ(Vₜ,Eₜ). It then obtains a new neighboring subnetwork (Vₜ₊₁,Eₜ₊₁) by randomly selecting X elements in the pruning vector P, and changing them, where X is proportional to the temperature variable. The latter is a variable that decreases exponentially with each iteration. The algorithm continues to the new neighbor subnetwork if this subnetwork performs better than the current one, meaning, if ƒ(Vₜ,Eₜ)<ƒ(Vₜ₊₁,Eₜ₊₁). The algorithm may also continue to a new subnetwork that worsens the performances, based on a probability distribution with a scale proportional to the temperature. By accepting subnetworks that lower the objective, the algorithm avoids being trapped in local maxima in early iterations and is able to explore globally for better solutions. The SA algorithm stops after 1-2000 iterations (for example, 500 iterations), and returns the best subnetwork found.

In some embodiments, the SA process may be employed 1-2000 times. For example, the process may be employed 1,000 times, resulting in 1,000 subnetworks. Each time the clinical drug response data is randomly divided into train and test sets. Both sets consist of the data describing 50% of the patients, preserving the patient-drug and cancer-type distributions of the original clinical drug response data. The training data is used strictly to construct the network, meaning to compute ƒ(V,E). The test set is used strictly to examine the solution, meaning the subnetwork that was selected according to a single SA run. If this subnetwork does not perform well on the test set, it is discarded. The different SA solutions are then aggregated to obtain the final SL-network. To this end SLICK weights each (A,B)εE_{I}, such that W_{F,(A,B)} denotes the fraction of SA solutions

$$A {\to }^{SL} B$$

appeared in. The final SL-network consists of SLi with a W_{F,(A,B)}≧C₂. Here C₂ was set to 0.1. This process enables SLICK to avoid over-fitting to the clinical drug response data and reduce the effect of data outliers.

### SLICK Objective Function: Predicting Clinical Drug Response

According to some embodiments, the objective function that guides the network optimization step estimates the ability of a potential SL-network to predict clinical drug response. It utilizes clinical drug response data that includes the gene expression profiles of cancer patients and information regarding the drugs these patients received. Let sample i be the clinical cancer sample derived from patient i. First, the network is integrated with the gene expression data to predict gene essentiality in each sample. The predicted essentiality of gene B in sample i is defined as the number of inactive SL-partners gene B has in this sample

Ess_{B,i}=Σ_{(A,B)εSLs}Inactive_{A,i} (6)

where Inactive_{A,i} is 1 if gene A is underexpressed in sample i, and 0 otherwise. The higher the Ess score of a gene is the more essential the gene is predicted to be. Gene essentiality predictions are then mapped to drug response predictions based on the mapping between drugs and their targets⁴⁴. The efficacy of a drug in a sample i is predicted as the median predicted essentiality of its targets in sample i. The response of patient i to a combination of drugs is predicted as the median efficacy predicted for these drugs in sample i.

In some embodiments, according to these predictions, the patients are classified as responders or non-responders if their predicted response to the drugs they received is in the top or bottom third of the drug response predictions obtained across all patients, respectively. The predictions are tested by examining if the patients that were classified as responders outlived the patients predicted as non-responders. This is done first when considering all patients and all drugs, obtaining a logrank p-value denoted as Pₐₗₗ. Next, the prediction is preformed and tested when considering only one drug at a time, for drugs that were received by at least 100 patients in the data. The different logrank p-values obtained separately for the different drugs are merged based on Fisher's combined probability test to obtained another p-value called P_{singleDrugs}. The final score is

$$s = \frac{( {P}_{all} + {P}_{singleDrugs} )}{2} \qquad ( 7 )$$

However, the above may merely test whether the network is predictive of patient survival. For example, consider a network consisting of false SLi between oncogenes, such a network is also likely to significantly discriminate between patients with high and low survival rates. Hence, the given network is also applied to predict drug response with a random patient-drug mapping, obtaining another score as described above, denoted as s_{random}. Importantly, the random mapping preserves the distribution of the patients across the different cancer types and drugs. Each SA iteration uses a different random patient-drug mapping as a control. The overall score of the network is the log ratio between its performances with the random mapping, and its performances with the true mapping:

$$f ( V , E ) = log ( \frac{{s}_{random}}{s} ) \qquad ( 8 )$$

In some embodiments, a network that merely predicts patient survival is likely to succeed regardless of the patient-drug mapping, and obtain a low score. In some embodiments, the final score captures the ability of an SL-network to provide genetic signatures that predict the response of a patient to specific drugs, as opposed to general prognostic genetic signatures.

According to some embodiments, the present invention provides system and method of providing a personalized cancer treatment comprising utilization of the SLICK system (approach) for identifying the optimal treatment in a specific patient or in a subpopulation of patients having cancer.

According to some embodiments, the methods and system disclosed herein can be used to predict drug responses to a drug or combination of drugs.

According to some embodiments, the system and methods of the present invention provide repurposing known active ingredients for cancer therapy.

According to some embodiments, there is provided a method of treating cancer in a subject in need thereof, the method comprising providing an anticancer treatment, wherein the anticancer treatment is determined according to the systems and methods of the invention. In some embodiments, the anticancer treatment comprises a repurposed drug (i.e., a known active ingredient used according to a different indication compared to the original indication for which it was intended to be used). According to some embodiments, the anticancer treatment comprises a combination of one more drugs. In some embodiments, the combination is synergistic. In some embodiments, the combination is determined based on the systems and methods disclosed herein.

According to some embodiments, the system and methods of the present invention may further be used for identification of new drug targets for treating cancer.

According to some embodiments, the system and methods of the present invention may be utilized for predicting the likelihood that a patient's cancer will respond to a specific therapy is provided. In some embodiments, the systems and methods disclosed herein may be used to determine responders and non-responders.

According to some embodiments, pharmaceutical composition comprising active agent according to the present invention may be administered as a stand-alone treatment or in combination with a treatment with any anti-neoplastic (anti-cancer) agent.

According to some specific embodiments, the anti-cancer composition comprises at least one chemotherapeutic agent. The chemotherapeutic agent, which could be administered separately or together with an agent according to the present invention, may comprise any such agent known in the art exhibiting anti-cancer activity, including but not limited to: mitoxantrone, topoisomerase inhibitors, spindle poison vincas: vinblastine, vincristine, vinorelbine (taxol), paclitaxel, docetaxel; alkylating agents: mechlorethamine, chlorambucil, cyclophosphamide, melphalan, ifosfamide; methotrexate; 6-mercaptopurine; 5-fluorouracil, cytarabine, gemcitabin; podophyllotoxins: etoposide, irinotecan, topotecan, dacarbazin; antibiotics: doxorubicin (adriamycin), bleomycin, mitomycin; nitrosoureas: carmustine (BCNU), lomustine, epirubicin, idarubicin, daunorubicin; inorganic ions: cisplatin, carboplatin; interferon, asparaginase; hormones: tamoxifen, leuprolide, flutamide, and megestrol acetate. According to a specific embodiment, the chemotherapeutic agent is selected from the group consisting of alkylating agents, antimetabolites, folic acid analogs, pyrimidine analogs, purine analogs and related inhibitors, vinca alkaloids, epipodopyllotoxins, antibiotics, L-asparaginase, topoisomerase inhibitor, interferons, platinum coordination complexes, anthracenedione substituted urea, methyl hydrazine derivatives, adrenocortical suppressant, adrenocorticosteroides, progestins, estrogens, antiestrogen, androgens, antiandrogen, and gonadotropin-releasing hormone analog. According to another embodiment, the chemotherapeutic agent is selected from the group consisting of 5-fluorouracil (5-FU), leucovorin (LV), irenotecan, oxaliplatin, capecitabine, paclitaxel and doxetaxel. Two or more chemotherapeutic agents can be used in a cocktail to be administered in combination with administration of the antibody or fragment thereof.

According some embodiments, the invention provides a method of treating cancer in a subject, comprising administering to the subject effective amount of an active agent identified by any of the methods of the present invention.

According to some embodiments, the cancer amenable treatment by the present invention includes, but is not limited to: carcinoma, lymphoma, blastoma, sarcoma, and leukemia or lymphoid malignancies. More particular examples of such cancers include squamous cell cancer, lung cancer (including small-cell lung cancer, non-small cell lung cancer, adenocarcinoma of the lung, and squamous carcinoma of the lung), cancer of the peritoneum, hepatocellular cancer, gastric or stomach cancer (including gastrointestinal cancer), pancreatic cancer, glioblastoma, cervical cancer, ovarian cancer, liver cancer, bladder cancer, hepatoma, breast cancer, colon cancer, colorectal cancer, endometrial or uterine carcinoma, salivary gland carcinoma, kidney or renal cancer, liver cancer, prostate cancer, vulval cancer, thyroid cancer, hepatic carcinoma and various types of head and neck cancer, as well as B-cell lymphoma (including low grade/follicular non-Hodgkin's lymphoma (NHL); small lymphocytic NHL; intermediate grade/follicular NHL; intermediate grade diffuse NHL; high-grade immunoblastic NHL; high-grade lymphoblastic NHL; high-grade small non-cleaved cell NHL; bulky disease NHL; mantle cell lymphoma; AIDS-related lymphoma; and Waldenstrom's Macroglobulinemia); chronic lymphocytic leukemia (CLL); acute lymphoblastic leukemia (ALL); Hairy cell leukemia; chronic myeloblastic leukemia; and post-transplant lymphoproliferative disorder (PTLD), as well as abnormal vascular proliferation associated with phakomatoses, edema (such as that associated with brain tumors), and Meigs' syndrome. Preferably, the cancer is selected from the group consisting of breast cancer, colorectal cancer, rectal cancer, non-small cell lung cancer, non-Hodgkins lymphoma (NHL), renal cell cancer, prostate cancer, liver cancer, pancreatic cancer, soft-tissue sarcoma, Kaposi's sarcoma, carcinoid carcinoma, head and neck cancer, melanoma, ovarian cancer, mesothelioma, and multiple myeloma. The cancerous conditions amendable for treatment of the invention include metastatic cancers.

In some embodiments, the present invention provides a method for increasing the duration of survival of a subject having cancer, comprising administering to the subject effective amount of a composition comprising an active agent identified by the present invention.

In some embodiments, the present invention provides a method for increasing the progression free survival of a subject having cancer, comprising administering to the subject effective amount of a composition comprising an active agent identified by any of the methods of the present invention.

According to some embodiments, the present invention provides a method for treating a subject having cancer, comprising administering to the subject effective amounts of a composition comprising an active agent identified by any of the methods of the present invention.

According to some embodiments, the present invention provides a method for increasing the duration of response of a subject having cancer, comprising administering to the subject effective amount of a composition comprising an active agent identified by any of the methods of the present invention.

In some embodiments, the present invention provides a method of preventing or inhibiting development of metastasis in a patient having cancer, comprising administering to the subject effective amounts of a composition comprising an active agent identified by any of the methods of the present invention.

According to some embodiments, there is provided a method of treating a disease or condition in a subject in need thereof, wherein the treatment regime (i.e., the type of drug, combination of drugs, dosage regime, administration mode, and the like) is determined according to the methods and systems disclosed herein.

According to some embodiments, there is provided a method of determining a treatment regime of a disease or condition in a subject in need thereof, wherein the treatment regime (i.e., the type of drug, combination of drugs, dosage regime, administration mode, and the like) is determined according to the methods and systems disclosed herein.

In some embodiments, the method comprises generating a suitable SLi network and applying the generated network on a genomic profile of cells obtained from the subject to determine the treatment regime.

### EXAMPLES

According to some exemplary embodiments, SLICK may be implemented in MATLAB, using the CONDOR distributed parallel computation infrastructure⁷³. SA searches apply the MATLAB built in SA function, with a maximum of 500 iterations, and an exponentially decreasing temperature function: T(t+1)=T (t)·0.95.

### Example 1. Applying SLICK to Construct the Clinical-SL-Network

Constructing the Protein-Coding Network.

The first step of p-values assignment was performed based on 19 datasets consisting of the SCNA, gene expression, somatic mutation profiles, and survival data of overall 4,764 clinical samples, spanning four cancer types: kidney renal clear cell carcinoma (506), ovarian serous cystadenocarcinoma (826), lung squamous cell carcinoma (448), and breast invasive carcinoma (2,984). The second step of network pruning and optimization was applied based on the gene expression, survival rates, and treatment information that was available for 1,471 TCGA cancer patients, spanning 23 cancer types, and overall 58 drugs. Nine out of these 58 drugs were given to more than 100 patients.

Constructing the miRNA Network.

SLICK was applied to identify the SLi of the form

$$miRNA {->}^{SL} protein \text{-} coding$$

gene. The first step of p-values assignment was performed based on 15 datasets consisting of the SCNA, miRNA and gene expression, somatic mutation profiles, and survival data of overall 1,883 clinical samples, spanning four cancer types: kidney renal clear cell carcinoma (268), ovarian serous cystadenocarcinoma (487), lung squamous cell carcinoma (343), and breast invasive carcinoma (785). The second step of network pruning and optimization was applied based on the miRNA expression, survival rates, and treatment information that was available for 1,568 TCGA cancer patients, spanning 27 cancer types, and overall 57 drugs. Eight of these 57 drugs were given to at least 100 patients.

### Predicting Gene Essentiality Based on SLi

The SL-network was applied to predict gene essentiality in cancer cell lines. First, Ess_{B,i} values (equation 6) are computed for every gene B and cell line i in the dataset, such that Inactive_{A,i} is 1 if gene A is strongly underexpressed and has a SCNA level below −0.1 in cell line i, and 0 otherwise. The predictions are then examined based on gene essentiality screens. A gene is labeled as essential in a certain cell line if its essentiality score in that cell line was among the top 1% essentiality scores obtained in the entire screen. Based on these labels and the gene essentiality predictions a Receiver Operating Characteristic (ROC) curve is computed for each cell line.

The ROC-curve plots the true positive rate vs. the false positive rate across all possible decision thresholds. The latter marks the minimal Ess_{B,i} value that is interpreted such that gene B is predicted to be essential in cell line i. The true positive rate, or sensitivity, is the fraction of correctly predicted essential genes out of the total number of essential genes. The false positive rate, also known as 1-specificity, is the number of nonessential genes that were falsely predicted as essential divided by the number of nonessential genes. The decision threshold starts from the highest and most stringent definition that results in a very small and top-ranked set of predicted essential genes, and moves towards a lower and more permissive setup in which more gene are predicted as essential. The area under the ROC-curve (AUC) is a conventionally used measure of the overall performance of a classifier, where an AUC of 0.5 denotes the performance of a random classifier and an AUC of 1 denotes the performance of an ideal classifier.

The clinical-SL-network was employed to predict gene essentiality in 129 cancer cell lines. For these cell lines both gene expression and SCNA data to were used to generate the predictions, and gene essentiality data for validation^{37,38,41}. To compare between DAISY and SLICK both networks were utilized as explained above to predict gene essentiality in each of the 129 cell lines. The 129 AUCs obtained by SLICK and those obtained by DAISY were compared by employing the one-sided Wilcoxon ranksum test.

### Predicting In-Vitro Drug Response Based on SLi

The SL-network was applied to predict in-vitro drug response based on the working hypothesis that a drug will be more effective in cell lines that underexpress the SL-partners of its drug targets. However, some of the drug target SL-partner may sensitize the cells to the drug only when their expression levels are negligible, while others may protect the cells from the drug cytotoxicity only when their expression is very high. Additionally, some SL-partners may be irrelevant in the in-vitro setting, or in the specific cancer types that were examined Unlike clinical drug response data, in-vitro drug response screens provide sufficient amounts of data to perform a supervised prediction process. This supervised process was employed only when testing the network predictions based on published drug response screens. All the novel predictions that were obtained for drug repurposing and drug combinations utilized the clinic-SL-network without any additional information or supervised learning.

The supervised prediction of in-vitro drug response was preformed separately for each drug, in a five-fold cross validation process. Let eff_{d,i} be the efficacies of drug d measured in cell line iεS, where S denotes all the cell lines examined in the screen. Divide the cell lines in S into five subsets, while preserving the distribution of eff_{d} and cancer types: S=∪_{c=1}⁵S_{c}. For each 1≦c≦5 let S_{c} be the test set, and Sₜᵣₐᵢₙ=S\S_{c} be the training set. Let Inactive_{A,i}, and PartiallyInactive_{A,i} be 1 if cell lines i underexpressed or strongly underexpressed gene A in cell i, respectively, and 0 otherwise. For every gene A which is an SL-partner of at least one of the drug targets compute two one-sided Wilcoxon ranksum test p-values: P1_{A} and P2_{A}. P1_{A} (P2_{A}) denotes whether the drug efficacies were significantly higher in cell lines that (strongly) underexpressed A compared to other cells, when considering only the cell lines in the training data. For every cell line j in the training set the response to drug d is predicted as

$${SL}_{{train}_{d , j}} = {\sum }_{P {1}_{A} < 0.05} {PartiallyInactive}_{A , j} + {\sum }_{P {2}_{A} < 0.05} {Inactive}_{A , j} \qquad ( 9 )$$

Similarly, for every cell line i in the test set the response to drug d is predicted as:

$${SL}_{{test}_{d , i}} = {\sum }_{P {1}_{A} < 0.05} {PartiallyInactive}_{A , i} + {\sum }_{P {2}_{A} < 0.05} {Inactive}_{A , i} \qquad ( 10 )$$

For each cell line in the test set we also predict the efficacy of drug d as

SL_{eff}_{_{d,i}}=E({eff_{d,j}|jεargmin_{jεS}_{ₜᵣₐᵢₙ}{|SLₜₑₛₜ_{_{d,i}}−SLₜᵣₐᵢₙ_{_{d,j}}|}}) (11)

After the cross validation process is completed SLₜₑₛₜ_{_{d,i}} and SL_{eff}_{_{d,i}} are available for every drug and every cell line in S. The predictions are examined for each drug by computing the Spearman and Pearson correlations between SLₜₑₛₜ_{_{d}} and eff_{d}. The predictions are also examined when considering all drugs and all cell lines by computing the correlations between SL_{eff} and eff.

The clinical-SL-network was applied as described above based on the Cancer Genome Project (CGP)³⁹, the Cancer Therapeutics Response Portal (CTRP)⁴⁰, and the Cancer Cell Line Encyclopedia (CCLE)⁴¹. The CGP data contains the IC50 values of 131 drugs across 639 cancer cell lines, where the IC50 of a drug denotes the drug concentration required to eradicate 50% of the cancer cells. The CTRP data includes the efficacies of 354 small molecules across 242 cancer cell lines. Drug efficacies in this case are quantified as the area-under-the-dose-curve. The CCLE data includes the efficacies of 24 drugs across 504 cell lines, where the efficacy is measured by the activity area, which is 1−the area-under-the-dose-curve. Gene expression profiles of 593 cell lines used in the CGP data were extracted from CGP, and the expression profiles of 241 and 470 cell lines used in the CTRP and CCLE screens, respectively, from CCLE⁴¹. The prediction was performed for all drugs that had at least one of their targets in the SL-network—107, 129, and 16 drugs included in the CGP, CTRP, and CCLE data, respectively. The drugs were mapped to their targets based on the mapping reported in CGP, CTRP, and DrugBank^{39,40,44}.

### Experimentally Testing the SL-Network as a Drug Repurposing Platform

Liv7k Cell Line.

HPV-negative cell line derived from a T3N2b primary tumor of the tongue. The patient received no chemo/radiotherapy prior to tumor excision. Generously gifted by Professor Richard Shaw (Head and Neck Cancer Consultant, Liverpool, UK). Cells are maintained in keratinocyte SFM (Life Technologies), supplemented with 2 mM L-glutamine, 0.2 ng/ml epidermal growth factor (EGF) and 25 μg/ml bovine pituitary extract (BPE). The cell line was tested from mycoplasma contamination and authenticated by exome sequencing and CNV.

Drug Repurposing Screen.

1397 compounds from the NIH Clinical Collection (Evotec, San Francisco, Calif.), LOPAC Pfizer (Sigma Aldrich), the Developmental Therapeutics Program (DTP)-Approved Oncology library and the FDA-Approved Drug Library (Selleck Chemicals) were tested at a single concentration of 10 μM. The libraries were received as 10 mM compounds in dimethyl sulfoxide (DMSO). Plates which conflicted with our plate layout were reformatted. The compounds were first diluted 1:50 in serum free media, and then further diluted 1:20 into to 96 well plates containing 5000 cells in 190 μl of keratinocyte SFM supplemented with 2 mM L-glutamine, 0.2 ng/ml EGF and 25 μg/ml BPE. 0.1% DMSO was used as a negative control, and staurosporine (Sigma Aldrich) at a concentration of 1 μM was used as a positive death control in all plates. Each drug was tested for 72 h under 21% and 0.1% O₂. The cells were fixed using 4% formaldehyde after 72 h, and stained with DAPI dilactate (Sigma Aldrich). To assess viability, stained nuclei were counted using Perkin Elmer Operetta High Content Microscope.

### Experimentally Testing the SL-Partners Predicted for BRCA and PARP

Pancreatic Ductal Adenocarcinoma (PDAC) Cells.

Capan1 (HTB-79) was derived from the liver metastasis of a Caucasian male PDAC patient. AsPC1-GFP (CRL-1682) was derived from the ascites metastasis of a Caucasian female PDAC patient. BxPC3-GFP (CRL-1687) was derived from the pancreas of a female PDAC patient. Patient derived xenograft cells (X50 and X57) were originated from ascites metastasis of PDAC patients (see below). Patient derived xenograft cells (X50) were authenticated by Promega PowerPlex 16 HS kit (BioRap Technology) by comparing to Germ-line DNA of matching patient. The cells have been tested for mycoplasma contamination using Biological Industries Mycoplasma Test Kit EZ-PCR (cat#20-700-20).

Xenograft Generation in Athymic Nude-Foxn1nu Mice.

All animal studies were approved by the Sheba Medical Center Institutional Animal Care and Use Committee (Helsinki 930/14). All patients have signed an informed consent and tissue samples were obtained following approval of the institutional ethical committee (Helsinki 4744 and 5539/13) and the Israeli Ministry of Health. Detailed xenograft generation method is currently under preparation for publication. In brief, ascites-derived PDAC cells and core-needle biopsies were collected following palliative paracentesis and diagnostic biopsies of PDAC metastatic tissues, respectively. Subsequently, the cells were subcutaneously transplanted into the flanks of 6-8 weeks old HSD nude mice. Xenografts were propagate, while tumors reaching ˜1.5 cm³ were harvested for further serial passaging in-vivo and digested to single cell suspension for primary cell culture generation.

Sulforhodamine B (SRB) Cytotoxicity Assays.

SRB assays were performed according as previously described⁷⁷. In brief, pancreatic cancer cells (Capan1, AsPC1, BxPC3 and PDX-derived cells) were seeded at a density of 4×10³ cells/well in 96 well plates in complete medium. After 24h of incubation they were subsequently treated with several concentrations and combination of PARP1/2/3 inhibitor (olaparib, Astra Zeneca, 0.25-8 μM), HDAC inhibitor (Vorinostat, Merck, 0.25-8 nM) and topoisomerase II inhibitor (VP-16, Ebewe, 0.25-8 nM). Additional drugs, listed in Table 1 were administered at single concentration that was chosen according to preliminary experiments. The experiment was conducted in triplicates. Plates were incubated at 37° C. in CO² incubator. After 72h cells were fixed with 10% trichloroacetic acid, stained with SRB and analyzed for percent of survival on a 96-well plate reader. The absorbance was measured using microplate reader at 540 nm.

Table 1. The selectivity of different drugs against the BRCA-deficient cell line Capan-1 compared to other pancreatic cancer cell lines which are wild type for BRCA.
The Fisher's combined p-values denoting whether the pertaining drug is selectively lethal to the BRCA-deficient Capan-1 cells compared to other cell lines. Olaparib—the positive control—is clinically established as selectively lethal to BRCA-deficient tumors. Etoposide (V16) was predicted by the SL-network as selectively lethal to BRCA-deficient cancer cells and tumors. Other drugs were tested as negative controls.

TABLE 1

| | | Capan-1 vs. other |
|---|---|---|
| Drugs | | cell-lines |
| Positive control | Olaparib | 2.68E−07 |
| SLi prediction | Etoposide (VP16) 1 NM | 9.52E−08 |
| (BRCA-TOP2A) | | |
| Negative controls | VINBLASTINE 0.2 NM | 0.068 |
| | YM155 10 NM | 0.500 |
| | VORINOSTAT 1 UM | 0.404 |
| | RITUXIMAB 10 NM | 0.053 |
| | RITUXIMAB 100 NM | 0.103 |
| | BORTEZOMIB 1 NM | 0.296 |
| | 5FU 1 UM | 0.408 |
| | IRINOTECAN 0.1 UM | 0.191 |
| | CARFILZOMIB 2 NM | 0.332 |
| | TRASTUZUMAB 6 NM | 0.251 |
| | PEMETREXED 20 NM | 0.500 |
| | CETUXIMAB 0.1 UM | 0.500 |
| | YM155 1 NM | 0.402 |
| | TRASTUZUMAB 0.6 NM | 0.398 |
| | YM155 0.1 NM | 0.494 |
| | GEMCITABINE 1 NM | 0.459 |

### Statistical Analysis of Drug Interactions.

One way by which an SLi

$$A {\to }^{SL} B$$

can be examined is by testing if the effect of a drug D_{A} that inhibits A is synergistic with the effect of drug D_{B} that inhibits gene B. The combination of two genes is defined as synergistic if it displays a stronger effect than expected based the effect observed when administering each of the two drugs separately. This concept is very similar to the approach used in genetic interaction screens²⁸ (see equation (1)). More specifically, cells are exposed to D_{A} and D_{B}, either alone or in combination. After a fixed time cellular viability is assessed and compared to an untreated control. For each pair of drugs the residual viability in each cell line is measured with n repeats

$${v}_{A , B , k} = {\{ {v}_{A , B , k}^{i} \}}_{1 \leq i \leq n} = {\{ \frac{ \begin{matrix}viability after treatment with {D}_{A} \\ and {D}_{B} in cell line k in experiment i\end{matrix} }{mean viability of vehicle - treated control in cell line k} \}}_{1 \leq i \leq n} \qquad ( 12 )$$

Similarly, for each drug its effect on the viability of each one of the cell lines is also measured

$${v}_{A , k} = {\{ {v}_{A , k}^{i} \}}_{1 \leq i \leq n} = {\{ \frac{ \begin{matrix}viability after treatment with {D}_{A} \\ in cell line k in experiment i\end{matrix} }{mean viability of vehicle - treated control in cell line k} \}}_{1 \leq i \leq n} \qquad ( 13 )$$

Given the measured residual viability of D_{A} and D_{B} the expected viability according to Bliss model of independence⁴⁷ is

Bliss_{A,B,k}={vⁱ_{A,k}·vⁱ_{B,k}}_{1≦i≦n,1≦J≦n} (14)

To test the synergy between two drugs in a specific cell line k a one-sided t-test is performed, examining whether the observed viabilities v_{A,B,k} are significantly lower than the expected viabilities in cell line k (Bliss_{A,B,k}). For each pair of drugs the t-test p-values computed for it are combined via Fisher's combined probability test. The combined p-value denotes the overall significance of the predicted synergy. To examine whether the entire set of predicted synergistic drug pairs is indeed synergistic, all the t-test p-values that were obtained for all drug combinations across all cell lines are combined via Fisher's combined probability test.
For drug combinations that were examined across several concentrations the synergy values were calculated also according to the Chou-Talalay method⁴⁹, using the CompuSyn software (ComboSyn, Inc., Paramus, N.J.). In this case the drug interaction is quantified by CI values, where CI<1, CI=1, and CI>1 indicate synergistic, additive, and antagonistic effects, respectively.

### RNAseq Procedure Details.

RNA extraction was made using Trizol. RNAseq profiles were obtained via the Illumina TruSeq RNA library, at a sequencing mode of SR60 (v4). Reads for each sample were mapped independently using TopHat version (v2.0.10)⁷⁸ against the human genome build hg19. Only uniquely mapped reads were used to determine the number of reads falling into each gene with the HTSeq-count package (0.6.1p1)⁷⁹. Raw count reads data was normalized based on DESeq2 package (v1.6.3)⁸⁰. For the RPKM calculation, the length of each gene is determined by the total exon length in base pair. The RPKM values of the two samples were normalized via quantile normalization, and compared when considering different groups of genes via paired t-tests.

### Example 2. The Clinical SL-Network Predicts In-Vitro Gene Essentiality and Drug Response

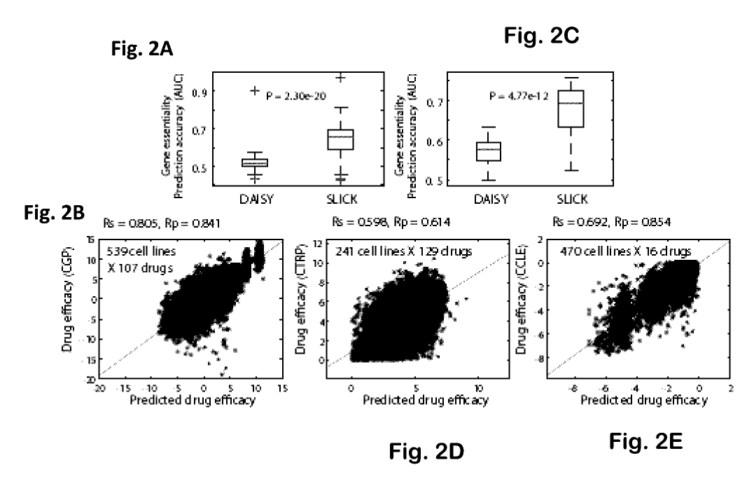

It was previously shown that an SL-network can be applied to predict gene essentiality in cancer cell lines³⁰. This is done by first analyzing the gene expression and SCNA profiles of the cancer cell line to identify inactive genes. The SL-essentiality-level of a gene in a given cell line is then defined as the number of inactive SL-partners this gene has in the pertaining cancer cell line according to the network³⁰. The clinical-SL-network was applied to predict gene essentiality in 129 cancer cell lines and the predictions were examined based on two gene essentiality screens^{37,38}. The clinical-SL-network obtained highly accurate predictions and outperformed the SL-network constructed by DAISY (FIGS. 2A and 2C, Wilcoxon rank-sum test p-value of 1.706e-28).

The clinical-SL-network was then applied to predict drug response in cancer cell lines. The sensitivity of a cell line to a given drug is predicted as proportional to the number of underexpressed SL-partners the drug target(s) have in that cell line, involving a supervised learning process. The network performances in this task were tested based on three drug response datasets consisting of the efficacies of overall 264 drugs across 879 cancer cell lines³⁹⁻⁴¹. The predicted drug efficacies are highly correlated to the measured drug efficacies according to all three screens (Pearson R=0.841, 0.614 and 0.854, P<1e-30, FIGS. 2B, 2D and 2E), again outperforming DAISY reported performance³⁰. Furthermore, the network successfully predicts the differential effect of a given drug across the cell lines for 145 drugs.

Focusing on novel predictions obtained by the clinical-SL-network three different and complementary experimental systems were set up to examine de novo whether the network can: (1) predict drug response and provide valuable leads for drug repurposing, (2) identify effective treatments for well-defined subpopulations of cancer patients, and (3) discover synergistic drug combinations.

### Example 3. Experimentally Testing the Clinical SL-Network as a Drug Repurposing Platform

An experimental screen was designed for testing whether the clinical SL-network can discriminate between cytotoxic and non-cytotoxic drugs, when considering a wide range of oncology and non-oncology drugs. Transcriptomic profiles of an oral cancer cell line under normoxia and hypoxia were obtained. Based on these profiles the network predicted the cell line response to 139 oncology and 531 non-oncology drugs in each condition. Then the predictions were proceeded to experimentally test by administering each of these 670 drugs at a concentration of 10 μM to the cells under hypoxia and normoxia.

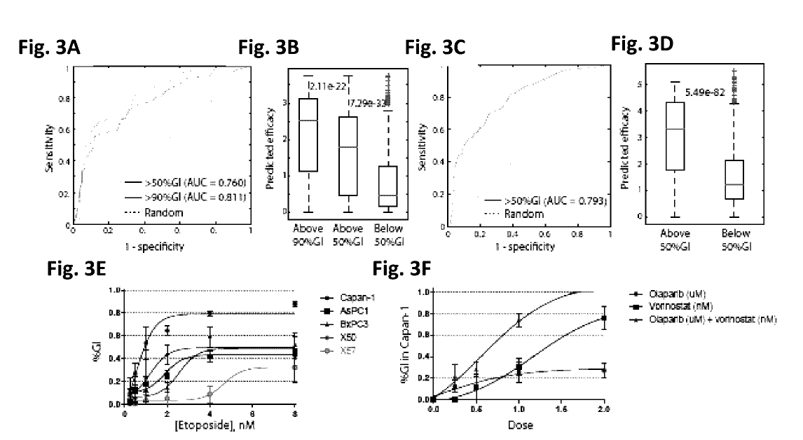

The predicted efficacies of the drugs matched the experimental findings: AUC=0.811 and 0.760, Wilcoxon ranksum p-values of 2.11e-22 and 7.29e-33, when defining drugs with more than 90% or 50% Growth Inhibition (GI) as effective, respectively (FIGS. 3A-3B). One of the most lethal repurposing candidate drugs according to the predictions is the antifungal drug terbinafine that inhibits squalene monooxygenase (SQLE). SQLE catalyzes the rate limiting step of sterol biosynthesis. Indeed, the administration of SQLE-inhibitors resulted in 81.9% GI under normoxia (IC50 of 5.8 μM). In line with the network predictions, SQLE-inhibition is less effective under hypoxia, resulting in 45.2% GI. To further validate the toxicity of SQLE-inhibition SQLE was inhibited via siRNA, resulting in 83.3% and 63.0% GI under normoxia and hypoxia, respectively.

The network ability to predict in-vitro drug response was then examined in seven breast cancer cell lines of different subtypes to 350 drugs. The majority of these drugs (323) are not approved to treat cancer. Based on the gene expression profiles of these cell lines⁴¹ the network accurately predicted their response to the drugs tested (AUC=0.793, Wilcoxon ranksum p-value of 5.490e-82, FIGS. 3C-3D).

### Example 4. Experimentally Testing the SL-Partners Predicted for BRCA and for PARP

The first and most successful SL-based therapy in cancer so far has been the treatment of BRCA1/2-deficient patients with PARP inhibitors. Although this approach has led to impressive clinical responses, multiple resistance mechanisms have been identified. The clinical SL-network was utilized to study the synthetic lethality associated with BRCA and PARP and explore their therapeutic potential.

The first objective has been to identify additional drugs that could selectively target BRCA-deficient tumors. The clinical-SL-network includes 126 and 22 SL-partners of BRCA1 and 2, respectively. Eleven of them are targeted by FDA-approved drugs, including the topoisomerase inhibitor etoposide⁴⁴. Etoposide was examined to three pancreatic cancer cell lines (including Capan-1—the only BRCA-deficient pancreatic cancer cell line) and two pancreatic cancer xenograft-derived cultures (wild type for BRCA1/2). As identified by the SLICK method, etoposide was substantially more cytotoxic in the Capan-1 BRCA-deficient cell line compared to the other cells (Fisher p-value<1e-30, FIG. 3E). To ensure this effect was not due to some generic susceptibility of Capan-1 to cytotoxic drugs its sensitivity to 12 other oncology drugs that were predicted as non-selective against BRCA-deficient cells were examined None of these drugs showed a selective effect (Table 1). These findings exhibit the validity and strength of the method of the present invention, reinforcing previous results that demonstrated the induced sensitivity of BRCA-deficient cells to topoisomerase inhibitors.

The clinical-SL-network includes 6, 1, and 283 SL-partners of PARP1/2/3 genes, respectively, 8 of which are targeted by oncology drugs. Among the predicted PARP SL-partners PLK1, RFC4 and TPX2 were found, whose siRNA knockdown was previously shown to sensitize cancer cells to PARP-inhibition^{11,13}. The clinical potential of PARP SLi was tested in two complementary ways. First, it was examined whether targeting PARP-SL-partners further sensitizes the cells to olaparib. Ten oncology drugs that inhibit the predicted PARP-SL partners were selected, and administered to the pancreatic cancer cell lines described above with or without olaparib. Overall strong synergistic effect between these drugs and olaparib was found (Fisher p-value of 2.453e-14, according to the Bliss independence model, Table 2)^{47,48}.

TABLE 2
The synergy between olaparib and drugs that target the predicted SL-partners of PARP3.
The p-values denote whether the observed efficacies of the drug combinations (olaparib and the
drugs listed in column A) are significantly more lethal compared to the expected effect, when considering
all cancer cell lines and cultures.

| | Fisher | | | | SLi | |
|---|---|---|---|---|---|---|
| | combined | | | | (maximal | Bidirectional |
| Drug | p-value | Targets | SLi | SLi (weight) | weight) | SLi |
| VORINOSTAT | 5.55E−09 | HDAC1, HDAC2, HDAC3, | HDAC2 + PARP10, HDAC2 + | 0.66262, 0.67324, | 0.805 | 1 |
| 1 μM | | HDAC6, HDAC8 | PARP3, PARP3 + HDAC2 | 0.80516 | | |
| IRINOTECAN | 5.13E−04 | TOP1, TOP1MT | BRCA1 + TOP2A, PARP3 + | 0.23427, 0.73237 | 0.732 | 0 |
| 0.1 μM | | | TOP2A | | | |
| GENCITABINE | 3.63E−03 | CMPK1, RRM1, TYMS | CMPK1 + BRCA1, PARP3 + | 0.12889, 0.2047 | 0.205 | 0 |
| 1 NM | | | TYMS | | | |
| BORTEZOMIB | 3.70E−03 | PSMB1, PSMB2, PSMB5, | PARP3 + PSMB5, PARP3 + | 0.10235, 0.39879 | 0.399 | 0 |
| 1 NM | | PSMD1, PSMD2 | PSMD2 | | | |
| VP16 1 NM | 7.41E−03 | TOP2A, TOP2B | BRCA1 + TOP2A, PARP3 + | 0.23427, 0.73237 | 0.732 | 0 |
| | | | TOP2A | | | |
| PEMETREXED | 1.47E−02 | ATIC, DHFR, GART, | PARP3 + TYMS | 0.2047 | 0.205 | 0 |
| 20 NM | | TYMS | | | | |
| YM155 1 NM | 4.80E−02 | BIRC5 | PARP3 + BIRC5 | 0.80136 | 0.801 | 0 |
| CARFILZOMIB | 9.18E−02 | PSMB1, PSMB2, PSMB5 | PARP3 + PSMB5 | 0.10235 | 0.102 | 0 |
| 2 NM | | | | | | |
| 5FU 1 UM | 4.51E−01 | TYMS | PARP3 + TYMS | 0.2047 | 0.205 | 0 |
| VINBLASTINE | 7.38E−01 | JUN, TUBA1A, TUBB, | PARP3 + TUBD1, PARP4 + | 0.15087, 0.26839 | 0.268 | 0 |
| 0.2 NM | | TUBD1, TUBE1, TUBG1 | TUBG1 | | | |

Among the SLi tested, the SLi with the strongest predicted weight was between PARP3 and HDAC2. Remarkably, the strongest synergism found experimentally is between olaparib and vorinostat, an inhibitor of HDAC2, with a median Combination Index (CI)⁴⁹ of 0.551 (a CI<0.9 denotes synergism, FIG. 3F). Of note, previous studies have shown the synergism between vorinostat and olaparib in triple-negative breast cancer, leukemia and thyroid cancer⁵⁰⁻⁵². Interestingly, thirty two genes are predicted as SL with both PARP and BRCA1/2, one of them is TOP2A, which was tested. Inhibiting such genes together with PARP in BRCA-deficient tumors could potentially be a very potent approach. Examining this approach it was found that etoposide—the inhibitor of TOP2A—is synergistic with olaparib (median CI value of 0.718).

In another experiment, xenograft models were generated from two biopsies collected from a pancreatic cancer patient with a germline mutation for BRCA1 (BRCA15382insC). The patient was treated with chemotherapy and a PARP-inhibitor. The first biopsy was taken before treatment, and the second was performed after the patient progressed on treatment. Gene expression profiles were obtained from the xenografts and it was found that, as expected, the SL-partners of PARP and TYMS (targeted by the chemotherapy) were significantly upregulated in the resistant xenograft (paired t-test p-values of 1.66e-22 and 4.63e-05, respectively). These results suggest that the SL-partners predicted for PARP may play a part in desensitizing the tumor to PARP-inhibitors.

### Example 5. SL-Based Prediction of Drug Response in Patients

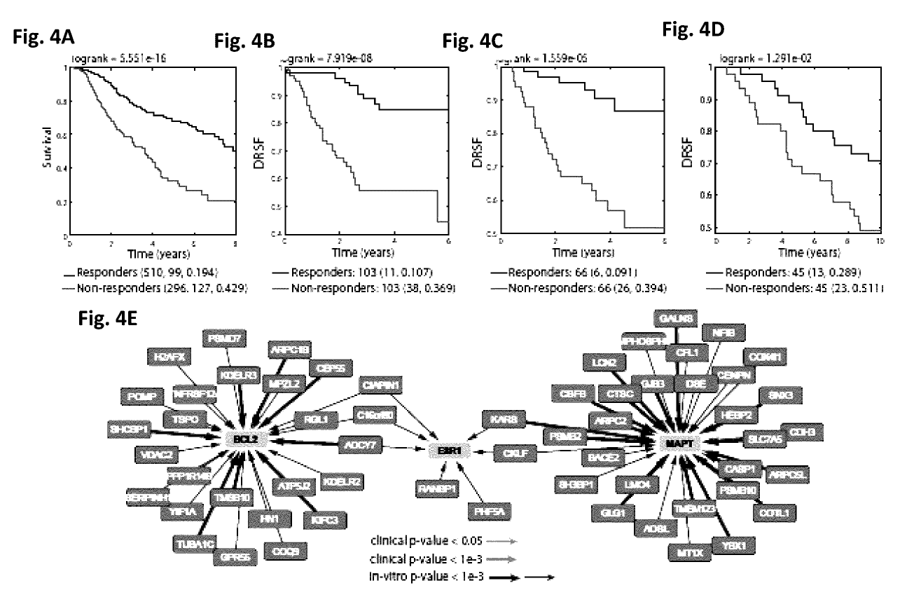

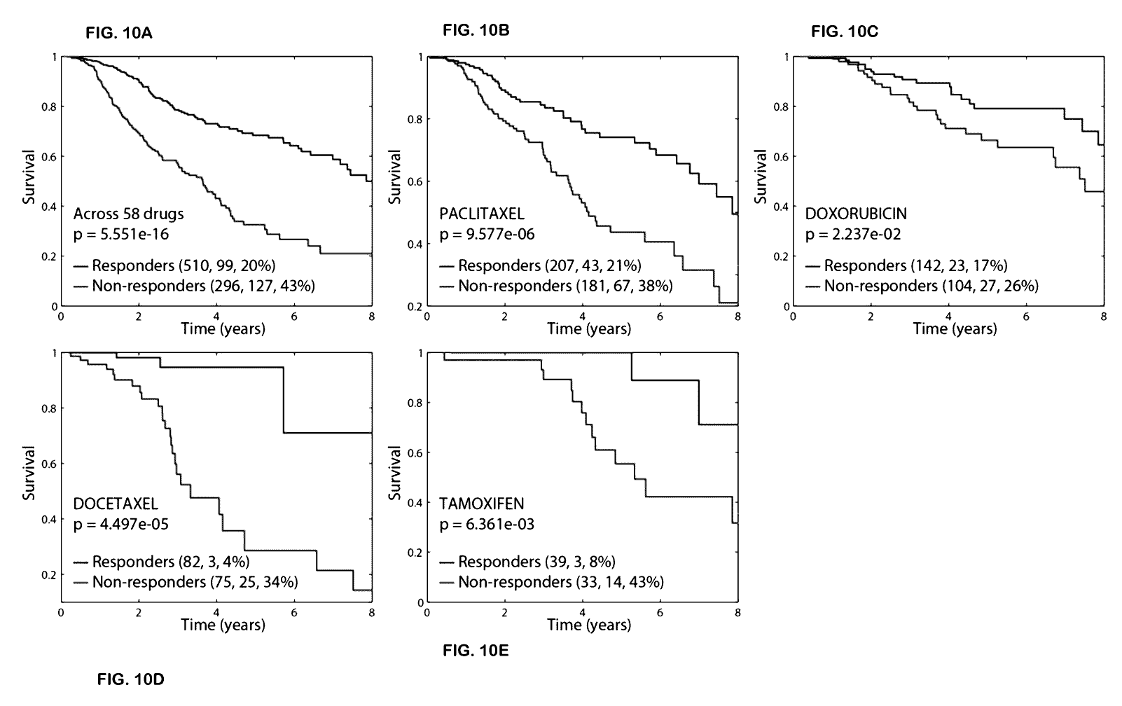

Going beyond cancer cell lines it was tested whether the clinical SL-network can predict drug response in cancer patients. First the TCGA drug response data was used in a cross validation process and it was found that patients classified as responders by the network outlived patients classified as non-responders (logrank p-value 5.55e-16, FIGS. 4A and 10). Reassuringly, the signal was lost when providing randomly shuffled mappings between patients and the drugs they received (empirical p-value<1e-3), testifying that the network predicts drug response beyond drug-independent patient survival. In contrast, DAISY method obtains statistically significant drug response predictions only for one drug (bevacizumab).

Next, the network was examined based on four independent clinical drug response datasets^{54,55}. The first two datasets were collected from overall 508 tumor biopsies of HER2-negative invasive breast cancer patients prior to treatment with neoadjuvant taxane-anthracycline chemotherapy⁵⁴. Patients that were classified by the clinical SL network as responders had significantly longer Distant Relapse-Free Survival (DRFS) rates compared to patients that were classified as non-responders (logrank p-value of 7.919e-08 and 1.559e-05, FIGS. 4B-4C). A positive correlation between the predicted strength of individual SLi and their ability to differentiate between responders and non-responders was also found in this dataset (Spearman R=0.228, P=1.579e-05). Notably, the SL-based predictors identified here are more predictive than the specific genetic drug response signatures identified in the original study (logrank P<1e-3 and P=2e-3)⁵⁴.

The third dataset included a well-annotated cohort of 207 breast cancer patients with complete 10-year follow-up⁵⁵. It consists of 134 estrogen receptor positive breast cancer patients treated with tamoxifen. The network obtained accurate drug response predictions in this cohort as well (logrank p-value of 1.29e-02, FIG. 4D). For comparison, the expression of ESR1 and ESR2 had no predictive signal (COX p-values of 0.606 and 0.567, respectively). Reassuringly, the tamoxifen response SL-predictor does not have a prognostic value when applied for breast cancer patients that were not treated with tamoxifen (logrank p-value of 0.117).

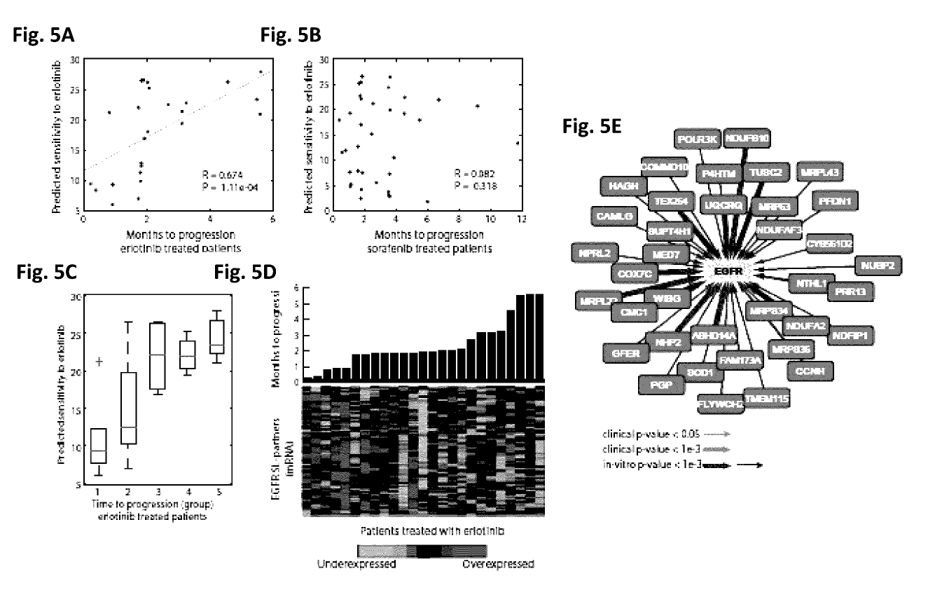

Lastly, the network was applied to predict the response of 25 patients with recurrent or metastatic Non-Small Cell Lung Cancer (NSCLC) to the EGFR-inhibitor erlotinib. All patients had EGFR wild-type tumors. As predicted, the number of underexpressed EGFR-SL-partners is a marker of longer time-to-progression (FIG. 5A, Spearman correlation R=0.674 and 0.656, P=1.11e-04 and 8.45e-04, when considering all patients or only KRAS wild-type patients, respectively). The predictions further improve when using only the strong SL-partners of EGFR (R=0.719 and 0.805, P=2.61e-05 and 7.67e-06, when using SLi with weights >0.25 and 0.5, respectively).

Aiming to identify the most valid SL-partners of EGFR the predictions were repeated while using one SLi at a time. Thirty seven of the 116 EGFR-SL-partners show a significant predictive signal (FIG. 5E), such that their predicted strength (weight) is correlated to their performance (R=0.350, P=6.291e-04). Additionally, 86.21% of the EGFR-SLi are supported by the response of cancer cell lines to EGFR-inhibitors, meaning that, their underexpression is significantly associated with higher efficacy of EGFR-inhibitors³⁹⁻⁴¹.

Next, it was tested whether the erlotinib SL-based response predictor indeed predicts the specific response to erlotinib, as opposed to merely predicting patient survival. Erlotinib response was predicted in an independent arm of the same trial in which 37 NSCLC patients were treated with sorafenib, a VEGFR inhibitor that is not represented in the network. Reassuringly, the predictions were not correlated to months-to-progression in this case (R=0.082 and P=0.318; FIG. 5B).

The performances of the clinical SL-network reported above improve upon those of other clinical drug response predictors applied previously to this data, which have achieved R=0.64 and 0.59, P=5.3e-04 and 6.4e-03, when applied to all patients or only KRAS wild-type patients⁵⁸. The original study reported a 76-gene epithelial-mesenchymal-transition signature with borderline ability to predict disease progression after eight weeks of treatment (t-test P=0.052)⁵⁶ compared to the SL predictor (t-test P=2.00e-04). The expression levels of EGFR itself do not convey a predictive signal (R=0.032 P=0.560). Additionally, even though mutated KRAS is a biomarker of resistance to EGFR-inhibitors⁵⁶, patients with KRAS mutations did not progress before KRAS wild-type patients (t-test P=0.384). DAISY derived networks failed to predict the clinical response to erlotinib (R≦0.157 and P≧0.227).

### Example 6. Identifying SLi Between miRNAs and Protein Coding Genes

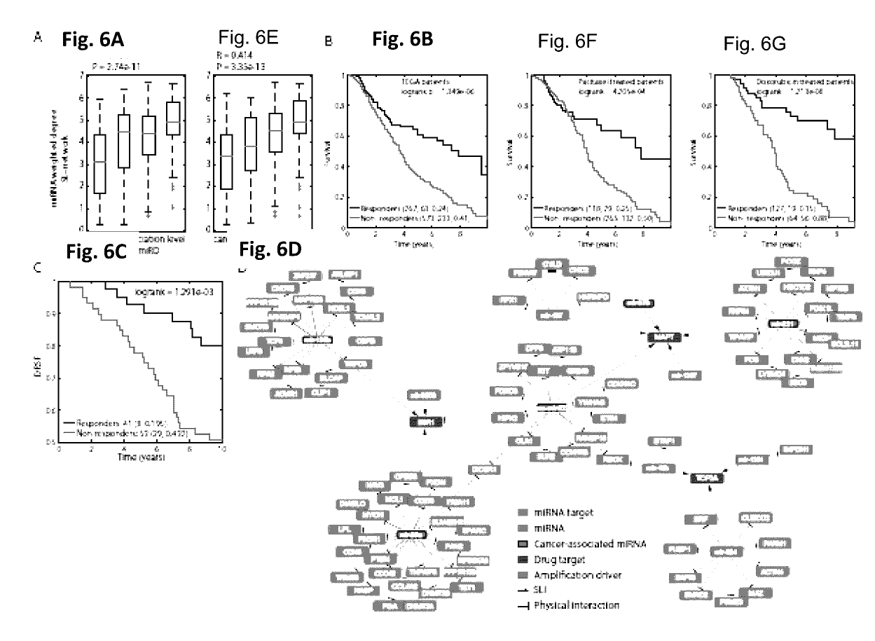

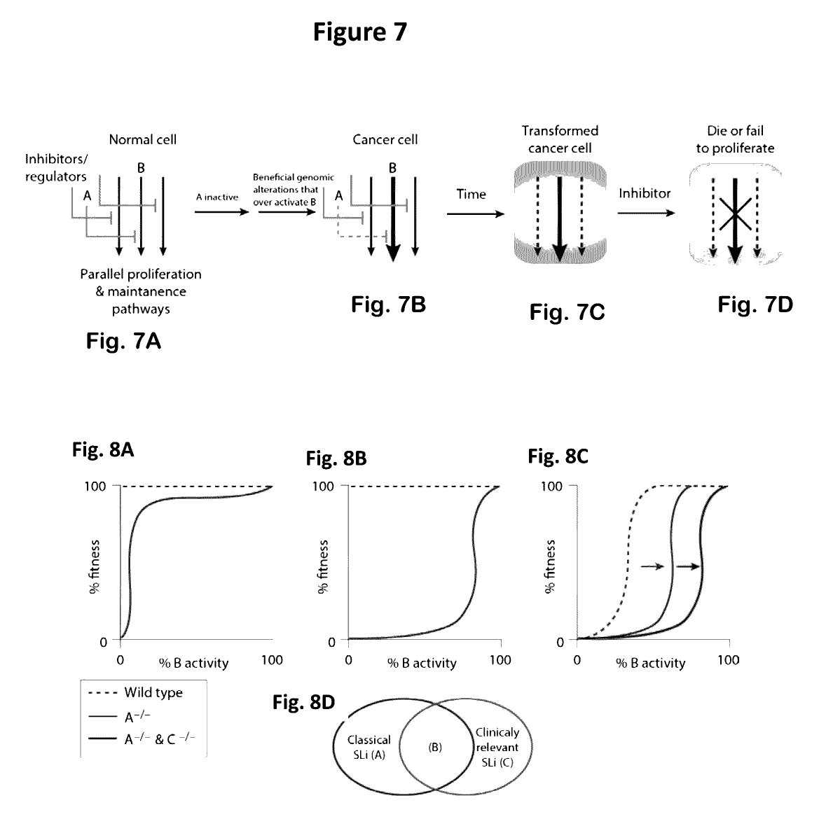

SLICK was applied to identify the SL-partners of micro-RNA (miRNA) genes, focusing on asymmetric SLi in which inactivation of a miRNA renders the cancer cells dependent on a protein coding gene. The resulting miRNA SL-network consists of 762 miRNAs, 12,612 protein-coding genes, and 116,020 SLi. Only a small fraction of the miRNA-SLi (2,219) are between miRNAs and their targets^{59,60}. miRNA hubs with many SLi include key tumor suppressors and oncogenes as mir-19a, mir-200c, let-7, and mir-145. Moreover, there is a significant correlation between the number of papers reporting the involvement of a given miRNA in cancer to its degree in the network (Spearman R=0.410 and 0.414, P=2.74e-11 and 3.35e-13, based on OncomiRDB⁶⁵ and miRCancer⁶⁶, respectively, FIGS. 6A and 6E). Linking between the regulatory role of miRNAs and their SLi, miRNAs with shared targets share significantly more SL-partners compared to miRNAs that have no shared targets (Wilcoxon ranksum p-value<1e-30).

The miRNA-SL-network accurately predicts clinical drug response when applied based on TCGA data in cross-validation, and the response of breast cancer patients to tamoxifen⁵⁵ (logrank p-values of 1.349e-06 and 1.291e-03, respectively, FIGS. 6B-6C and 6F-6G). Reassuringly, the predictive signal is lost on randomly-shuffled patient-drug maps (empirical p-value<1e-3). Likewise, the tamoxifen response predictor has border-line prognostic value when applied to patients that were not treated with tamoxifen (logrank p-value of 0.053). Aiming to identify the most valid and clinically relevant miRNA SLi the predictions were repeated while using one SLi at a time (the top most predictive SLi are shown in FIG. 6D). It was found that the response to the chemotherapies paclitaxel and doxorubicin is enhanced when miR-221 and miR-222 are downregulated. Indeed miR-221/2 have been shown before to promote chemoresistance, and plasma levels of miR-221 are predictive for chemoresistance.

### Example 6. Revising the Definition of Synthetic Lethal Interactions (SLi)

The notion of clinically-relevant Synthetic Lethal interactions (SLi) was defined. Consider the SLi between two genes, denoted as A and B. This SLi will have a pharmacological value only if the partial inhibition of B, and not only the complete inhibition of B, has a substantial effect in A-deficient cells (FIGS. 8A, 8B). This is because drugs often cause partial rather than complete inhibition of their targets. Furthermore the activity level of the drug target is expected to be fairly high in the treated cancer cells, due to the inactivity of their Synthetic Lethal (SL)-partner(s). Hence, it is even less likely that the drug will completely block its targets in these cells. Therefore, SL-Identification based on Clinical Knowledge (SLICK) uses a permissive definition of gene underexpression when examining whether the underexpression of the drug target SL-partners sensitize the cancer cells to the drug. In this manner SLICK is more likely to identify pharmacologically relevant SLi (FIG. 8B), rather than irrelevant ones (FIG. 8A).

Additionally, the classical definition of SLi, whereby the inhibition of a certain gene is lethal only to the mutant or deficient cells, covers only a fraction of the clinically-relevant SLi (FIG. 8B). Even if gene B is required for both A-deficient and for the wild-type cells, B-inhibitors could have a significant therapeutic index (FIG. 8C). It is sufficient to require that the inactivation of A shifts or alters the fitness dose-response curve of B-inhibitors such that keeping B activity below a certain threshold selectively impairs A-deficient cells. Moreover, the cumulative nature of some SLi can further extend the therapeutic window and induce drug selectivity and efficacy (FIG. 8C). The network optimization process employed by SLICK is specifically tailored to identify cumulative SLi.

### Example 7. The Clinical-SL-Network

### Regulatory SLi

A particularly interesting class of asymmetric SLi—that was termed regulatory SLi—captures tumorigenic events where following the inactivation of a tumor suppressor cancer cells become, over time, dependent on specific oncogenic transformations ( ). Indeed, it was found that regulatory SLi, meaning, asymmetric interactions of the form tumor suppressor→oncogene, are predicted as significantly stronger compared to other SLi (Wilcoxon ranksum p-values of 1.07e-79; tumor suppressors and oncogenes were defined according to tumorscape⁸²). Accordingly, tumor suppressors have significantly higher out-degrees and lower in-degrees compared to other genes (Wilcoxon ranksum p-values of 6.60e-35 and 1.18e-09, respectively), while oncogenes have significantly lower out-degrees and higher in-degrees (Wilcoxon ranksum p-values of 1.39e-16 and 1.77e-17, respectively). Thus, according to the SL-network the loss of tumor suppressors is likely to sensitize the cells to subsequent alterations and result in oncogene addictions (high out-degree), while the inhibition of oncogenes is likely to selectively kill cancer cells (high in-degree).

### BRCA-SLi

Additional noteworthy SL-partners that were predicted for BRCA1/2 include EZH2 that was predicted as SL with BRCA1. Previous studies showed that BRCA1-deficient cancer cells are selectively dependent on their elevated EZH2 levels, and that inhibiting EZH2 is ˜20-fold more effective in killing BRCA1-deficient cells compared to BRCA1-proficient mammary tumor cells. BRCA1/2 were also predicted as SL with RAD54L, RAD51AP1, and RAD51, which are involved in DNA double-strand break repair and homologous recombination. Supporting these predictions, RAD51 interacts with RAD52. The latter has been shown to be SL with BRCA1/2⁸⁶.

### REFERENCES

1. Hartwell, L. H., Szankasi, P., Roberts, C. J., Murray, A. W. & Friend, S. H. Integrating Genetic Approaches into the Discovery of Anticancer Drugs. Science 278, 1064-1068 (1997).
2. Brough, R., Frankum, J. R., Costa-Cabral, S., Lord, C. J. & Ashworth, A. Searching for synthetic lethality in cancer. Current Opinion in Genetics & Development 21, 34-41 (2011).
3. Ashworth, A., Lord, Christopher J. & Reis-Filho, Jorge S. Genetic Interactions in Cancer Progression and Treatment. Cell 145, 30-38 (2011).
4. Kaelin, W. The concept of synthetic lethality in the context of anticancer therapy. Nat Rev Cancer 5, 689-698 (2005).
5. Luo, J., Solimini, N. L. & Elledge, S. J. Principles of Cancer Therapy: Oncogene and Non-oncogene Addiction. Cell 136, 823-837 (2009).
8. Weinstein, I. B. & Joe, A. Oncogene Addiction. Cancer Research 68, 3077-3080 (2008).
9. Davoli, T. et al. Cumulative Haploinsufficiency and Triplosensitivity Drive Aneuploidy Patterns to Shape the Cancer Genome. Cell 155, 948-962 (2013).
10. Farmer, H. et al. Targeting the DNA repair defect in BRCA mutant cells as a therapeutic strategy. Nature 434, 917-921 (2005).
11. Lord, C. J., McDonald, S., Swift, S., Turner, N. C. & Ashworth, A. A high-throughput RNA interference screen for DNA repair determinants of PARP inhibitor sensitivity. DNA Repair 7, 2010-2019 (2008).
12. Martin, S. A. et al. Methotrexate induces oxidative DNA damage and is selectively lethal to tumour cells with defects in the DNA mismatch repair gene MSH2. EMBO Molecular Medicine 1, 323-337 (2009).
13. Turner, N. C. et al. A synthetic lethal siRNA screen identifying genes mediating sensitivity to a PARP inhibitor. EMBO J 27, 1368-1377 (2008).
14. Vizeacoumar, F. J. et al. A negative genetic interaction map in isogenic cancer cell lines reveals cancer cell vulnerabilities. Mol Syst Biol 9, 696 (2013).
15. Kessler, J. D. et al. A SUMOylation-Dependent Transcriptional Subprogram Is Required for Myc-Driven Tumorigenesis. Science 335, 348-353 (2012).
16. Luo, J. et al. A Genome-wide RNAi Screen Identifies Multiple Synthetic Lethal Interactions with the Ras Oncogene. Cell 137, 835-848 (2009).
17. Szamecz, B. et al. The Genomic Landscape of Compensatory Evolution. PLoS Biol 12, e1001935 (2014).
18. Chipman, K. & Singh, A. Predicting genetic interactions with random walks on biological networks. BMC Bioinformatics 10, 17 (2009).
19. Kelley, R. & Ideker, T. Systematic interpretation of genetic interactions using protein networks. Nat Biotech 23, 561-566 (2005).
20. Szappanos, B. et al. An integrated approach to characterize genetic interaction networks in yeast metabolism. Nat Genet 43, 656-662 (2011).
21. Wong, S. et al. Combining biological networks to predict genetic interactions. Proceedings of the National Academy of Sciences 101, 15682-15687 (2004).
22. Thong, W. & Sternberg, P. W. Genome-wide prediction of C. elegans genetic interactions. Science 311, 1481-4 (2006).
23. Lu, X., Kensche, P. R., Huynen, M. A. & Notebaart, R. A. Genome evolution predicts genetic interactions in protein complexes and reveals cancer drug targets. Nat Commun 4(2013).
24. Conde-Pueyo, N., Munteanu, A., Sole, R. & Rodriguez-Caso, C. Human synthetic lethal inference as potential anti-cancer target gene detection. BMC Systems Biology 3, 116 (2009).
25. Folger, O. et al. Predicting selective drug targets in cancer through metabolic networks. Mol Syst Biol 7(2011).
26. Frezza, C., Pollard, P. & Gottlieb, E. Inborn and acquired metabolic defects in cancer. Journal of Molecular Medicine 89, 213-220 (2011).
27. Wang, X. & Simon, R. Identification of potential synthetic lethal genes to p53 using a computational biology approach. BMC Med Genomics 6, 30 (2013).
28. Costanzo, M. et al. The Genetic Landscape of a Cell. Science 327, 425-431 (2010).
29. Boucher, B. & Jenna, S. Genetic interaction networks: better understand to better predict. Front Genet 4, 290 (2013).
30. Jerby-Arnon, L. et al. Predicting Cancer-Specific Vulnerability via Data-Driven Detection of Synthetic Lethality. Cell 158, 1199-1209 (2014).
31. Ryan, Colm J., Lord, Christopher J. & Ashworth, A. DAISY: Picking Synthetic Lethals from Cancer Genomes. Cancer Cell 26, 306-308 (2014).
32. Szczurek, E., Misra, N. & Vingron, M. Synthetic sickness or lethality points at candidate combination therapy targets in glioblastoma. International Journal of Cancer 133, 2123-2132 (2013).
33. Lu, X., Megchelenbrink, W., Notebaart, R. A. & Huynen, M. A. Predicting Human Genetic Interactions from Cancer Genome Evolution. PLoS ONE 10, e0125795 (2015).
34. Park, S. & Lehner, B. Cancer type-dependent genetic interactions between cancer driver alterations indicate plasticity of epistasis across cell types. Molecular Systems Biology 11, n/a-n/a (2015).
35. Ciriello, G., Cerami, E., Sander, C. & Schultz, N. Mutual exclusivity analysis identifies oncogenic network modules. Genome Res 22, 398-406 (2012).
36. Leiserson, M. M., Wu, H.-T., Vandin, F. & Raphael, B. CoMEt: A Statistical Approach to Identify Combinations of Mutually Exclusive Alterations in Cancer. in Research in Computational Molecular Biology, Vol. 9029 (ed. Przytycka, T. M.) 202-204 (Springer International Publishing, 2015).
37. Marcotte, R. et al. Essential Gene Profiles in Breast, Pancreatic, and Ovarian Cancer Cells. Cancer Discovery 2, 172-189 (2012).
38. Cheung, H. W. et al. Systematic investigation of genetic vulnerabilities across cancer cell lines reveals lineage-specific dependencies in ovarian cancer. Proceedings of the National Academy of Sciences (2011).
39. Garnett, M. J. et al. Systematic identification of genomic markers of drug sensitivity in cancer cells. Nature 483, 570-575 (2012).
40. Basu, A. et al. An Interactive Resource to Identify Cancer Genetic and Lineage Dependencies Targeted by Small Molecules. Cell 154, 1151-1161 (2013).
41. Barretina, J. et al. The Cancer Cell Line Encyclopedia enables predictive modelling of anticancer drug sensitivity. Nature 483, 603-307 (2012).
44. Knox, C. et al. DrugBank 3.0: a comprehensive resource for ‘Omics’ research on drugs. Nucleic Acids Research 39, D1035-D1041 (2011).
47. Bliss, C. I. THE TOXICITY OF POISONS APPLIED JOINTLY1. Annals of Applied Biology 26, 585-615 (1939).
48. Dietlein, F. et al. A Synergistic Interaction between Chk1- and MK2 Inhibitors in KRAS-Mutant Cancer. Cell 162, 146-59 (2015).
49. Chou, T. C. Drug combination studies and their synergy quantification using the Chou-Talalay method. Cancer Res 70, 440-6 (2010).
50. Adimoolam, S. et al. HDAC inhibitor PCI-24781 decreases RAD51 expression and inhibits homologous recombination. Proc Natl Acad Sci USA 104, 19482-7 (2007).
51. Baldan, F. et al. Synergy between HDAC and PARP Inhibitors on Proliferation of a Human Anaplastic Thyroid Cancer-Derived Cell Line. International Journal of Endocrinology 2015, 7 (2015).
52. Ha, K. et al. Histone deacetylase inhibitor treatment induces ‘BRCAness’ and synergistic lethality with PARP inhibitor and cisplatin against human triple negative breast cancer cells. Oncotarget 5, 5637-5650 (2014).
54. Hatzis, C., Pusztai, L., Valero, V. & et al. A genomic predictor of response and survival following taxane-anthracycline chemotherapy for invasive breast cancer. JAMA 305, 1873-1881 (2011).
55. Buffa, F. M. et al. microRNA-associated progression pathways and potential therapeutic targets identified by integrated mRNA and microRNA expression profiling in breast cancer. Cancer Res 71, 5635-45 (2011).
56. Byers, L. A. et al. An epithelial-mesenchymal transition gene signature predicts resistance to EGFR and PI3K inhibitors and identifies Axl as a therapeutic target for overcoming EGFR inhibitor resistance. Clin Cancer Res 19, 279-90 (2013).
58. Geeleher, P., Cox, N. J. & Huang, R. S. Clinical drug response can be predicted using baseline gene expression levels and in vitro drug sensitivity in cell lines. Genome Biol 15, R47 (2014).
59. Consortium, T. E. P. An Integrated Encyclopedia of DNA Elements in the Human Genome. Nature 489, 57-74 (2012).
60. Hsu, S.-D. et al. miRTarBase update 2014: an information resource for experimentally validated miRNA-target interactions. Nucleic Acids Research 42, D78-D85 (2014).
65. Wang, D., Gu, J., Wang, T. & Ding, Z. OncomiRDB: a database for the experimentally verified oncogenic and tumor-suppressive microRNAs. Bioinformatics 30, 2237-8 (2014).
66. Xie, B., Ding, Q., Han, H. & Wu, D. miRCancer: a microRNA-cancer association database constructed by text mining on literature. Bioinformatics 29, 638-644 (2013).
73. Thain, D., Tannenbaum, T. & Livny, M. Distributed computing in practice: the Condor experience: Research Articles. Concurr. Comput.: Pract. Exper. 17, 323-356 (2005).
77. Vichai, V. & Kirtikara, K. Sulforhodamine B colorimetric assay for cytotoxicity screening. Nat Protoc 1, 1112-6 (2006).
78. Kim, D. et al. TopHat2: accurate alignment of transcriptomes in the presence of insertions, deletions and gene fusions. Genome Biology 14, R36 (2013).
79. Anders, S., Pyl, P. T. & Huber, W. HTSeq—a Python framework to work with high-throughput sequencing data. Bioinformatics 31, 166-9 (2015).
80. Love, M., Huber, W. & Anders, S. Moderated estimation of fold change and dispersion for RNA-seq data with DESeq2. Genome Biology 15, 550 (2014).
82. Beroukhim, R. et al. The landscape of somatic copy-number alteration across human cancers. Nature 463, 899-905 (2010).
86. Feng, Z. et al. Rad52 inactivation is synthetically lethal with BRCA2 deficiency. Proc Natl Acad Sci USA 108, 686-91 (2011).

1. A system for identifying clinically relevant synthetic lethal interactions (SLi) of pairs of genes from cancer patients, the system comprising:
a non-transitory computer readable memory having stored thereon datasets comprising data related to multiple genes in said cancer patients, and
a processing circuitry configured to recursively:
i. assign SLi-p values to each ordered gene pair obtained from a cancer patient data (dataset x-gene x);
ii. omit unlikely SLi to obtain significant SLi-p values;
iii. perform simulated annealing (SA) to optimize the network ability to predict a clinical drug response;
iv. repeat step (iii) N times; and
v. merge the solutions obtained in (iv) to construct a final SL network according to all N solutions.

2. The system of claim 1, wherein the SLi-p-value denote the likelihood of a gene pair of being synthetic Lethal (SL).

3. The system of claim 2, wherein the SLi-p-value is determined utilizing data-driven inference procedures selected from: genomic Survival of the Fittest (gSoF); Clinical survival analyses, and/or Correlated expression.

4. The system of claim 1 wherein step (iii) comprises eliminating false positive predictions emerged in steps (i.) or (ii.).

5. The system of claim 1, wherein N is at least 200.

6. The system of claim 1 weight or strength of a given SLi in the final SL network is the fraction of solutions in which it appeared.

7. The system of claim 1 wherein the cancer patient data is selected from activity profile of the genes, essentiality profile of the genes, expression profile of the genes, treatment, response to treatment, prognosis, survival, or combinations thereof.

8. The system of claim 7 wherein activity profile of the genes comprises Somatic Copy Number of Alterations (SCNA), germline Copy-Number Variations (CNV), DNA methylation, histone methylation, somatic mutations, germline mutations or combinations thereof, obtained from at least one cancer patient.

9. The system of claim 1 wherein the processing circuitry is further configured to determine an occurrence selected from the group consisting of:
a. response of cancer cells to the inhibition of a gene product;
b. survival of a subject having cancer;
c. response of cancer cells to a specific drug; and
d. ranking of cancer treatments for a specific subject having cancer;
wherein the determination comprising applying the identified SL-network on a genomic profile of cells, wherein the genomic profile of cells is obtained from at least one cancer patient.

10. The system of claim 1, further comprising predicting one or more of: clinical response of a cancer patient to a drug; drug repurposing, drug combinations, or combinations thereof.

11. A system for predicting clinical anti-cancer drug response utilizing a clinically relevant synthetic lethal interactions (SLi) network of pairs of genes from cancer patients, the system comprising:
a non-transitory computer readable memory having stored thereon datasets comprising data related to multiple genes in said cancer patients, and a processing circuitry configured to recursively:
i. assign SLi-p values to each ordered gene pair obtained from a cancer patient data (dataset x-gene x);
ii. omit unlikely SLi to obtain significant SLi-p values;
iii. perform simulated annealing (SA) to optimize the network ability to predict a clinical drug response;
iv. repeat step (iii) N times;
v. merge the solutions obtained in (iv) to construct a clinically relevant synthetic lethal interactions (SLi) network according to all N solutions;
vi. integrating a SLi network of step (v.) 1 with a gene expression profile of at least one subject's tumor;
vii. predicting the response of said at least one subject tumor to a specific drug as proportional to the number of underexpressed SL-partners the specific drug target(s) has in the subject's tumor;
viii. classifying the subjects, based on step (vii) as responders or non-responders to the specific treatment said subjects received, and providing a computed logrank p-value to examine whether the responders outlived the non-responders;
ix. computing a control logrank p-value using randomly shuffled drug-patient mappings; and
x. calculating the ratio between the p-values of (viii) and (ix), wherein said ratio denotes the ability of the SL-network to specifically predict drug response while controlling for drug-independent patient survival rates.

12. The system of claim 11, wherein the SLi-p-value denote the likelihood of a gene pair of being synthetic Lethal (SL).

13. The system of claim 11, wherein step (iii) comprises eliminating false positive predictions emerged in steps (i.) or (ii.).

14. The system of claim 11, wherein N is at least 200.

15. The system of claim 11 comprising analyzing at least one data type selected from the group consisting of: Somatic Copy Number Alterations (SCNA), gene expression, somatic mutation profiles, treatment information, and survival data collected from clinical samples of subjects having cancer.

16. The system of claim 15 comprising analyzing at least one additional data type.

17. The system of claim 16 wherein the at least one additional data type is selected from the group consisting of: single-cell gene expression data, proteomics, protein modifications, and epigenetic alterations.

18. The system of claim 11, further comprising predicting drug repurposing and drug combinations useful in treating a subject's cancer condition.

19. A method of treating cancer in a subject having a BRCA1/2-deficient cancer comprising administering to said subject a combination therapy comprising olaparib and etoposide.

20. The method of claim 19 wherein olaparib and etoposide are administered separately or in a combined composition comprising olaparib and etoposide.

21. The method of claim 19 wherein the cancer is selected from the group consisting of:
oral cancer, breast cancer and pancreatic cancer.
